(19) United States
(12) Patent Application Publication
(10) Pub. No.: US 2010/0285446 A1
(43) Pub. Date: Nov. 11, 2010

(54) Methods for Detecting Metabolic States by Laser Ablation Electrospray Ionization Mass Spectrometry

(76) Inventors: Akos Vertes, Reston, VA (US); Fatah Kashanchi, Potomac, MD (US); Prabhakar Sripadi, Hyderabad (IN); Kylene Kehn-Hall, Fredericksburg, VA (US)

Correspondence Address: K&L GATES LLP, 210 SIXTH AVENUE, PITTSBURGH, PA, 15222-2613, US

(21) Appl. No.: 12/774,533

(22) Filed: May 5, 2010

Related U.S. Application Data
(63) Continuation-in-part of application No. 12/323,276, filed on Nov. 25, 2008.
Application No. 12/323,276 is a continuation-in-part of application No. 12/176,324, filed on Jul. 18, 2008.
(60) Provisional application No. 60/951,186, filed on Jul. 20, 2007.

(51) Int. Cl. C12Q 1/70; H01J 49/26; C12Q 1/68; C12Q 1/02

(52) U.S. Cl. 435/5; 250/282; 435/6; 435/29; 250/252.1

(57) ABSTRACT

According to certain embodiments, a method of mass spectrometry may generally comprise subjecting a sample comprising at least one indicator to laser ablation electrospray ionization mass spectrometry; determining a relative intensity of the indicator; and comparing the relative intensity of the indicator to a standard indicator intensity. Subjecting a sample to laser ablation electrospray ionization mass spectrometry may comprise ablating the sample with an infrared laser under ambient conditions to form an ablation plume; intercepting the ablation plume by an electrospray plume; and detecting the indicator by mass spectrometry. The method of mass spectrometry may comprise classifying the sample as belonging to or not belonging to the standard indicator intensity. A sample not belonging to the standard indicator intensity may indicate that the sample is predicted to comprise a disease state.

### CROSS-REFERENCE TO RELATED APPLICATIONS

This is a continuation-in-part of U.S. application Ser. No. 12/176,324, filed on Jul. 18, 2008, which claims priority to U.S. provisional application Ser. No. 60/951,186, filed on Jul. 20, 2007, and U.S. application Ser. No. 12/323,276, filed on Nov. 25, 2008, each of the foregoing applications are hereby incorporated herein by reference in their entireties.

### STATEMENT OF GOVERNMENTAL INTEREST

Portions of this invention were made with United States government support under Grant Nos. 0415521 and 0719232 awarded by the National Science Foundation. The government has certain rights in the invention.

### BACKGROUND

The apparatuses and methods described herein generally relate to ionization sources for mass spectrometers and methods of mass spectrometry, and in particular, laser ablation electrospray ionization mass spectrometry as well as methods of making and using the same.

Infectious diseases and metabolic disorders cause death, disability, and social and economic distress for millions of people throughout the world. Examples of the causative agents of infectious diseases include, but are not limited to, human immunodeficiency virus 1 (HIV-1), human immunodeficiency virus 2 (HIV-2), human T-lymphotropic virus type 1 (HTLV1),), human T-lymphotropic virus type 2 (HTLV2), human T-lymphotropic virus type 3 (HTLV3), human papillomavirus (HPV), hepatitis A, hepatitis B, hepatitis C, viral encephalitis, herpes virus, paramyxoviruses, influenza, and severe acute respiratory syndrome (SARS). Examples of metabolic disorders include, but are not limited to, diabetes, muscular dystrophy, phenylketonuria (PKU), Tay Sachs disease, leukodystrophies, lysosomal disorders, Wilson's disease, Lesch-Nyhan syndrome, urea cycle disorder, amyloidosis and lipid storage diseases. Genomic, proteomic, and metabolomic technologies have been used to identify biomarkers and other indicators of infectious diseases and metabolic disorders. Rapid monitoring of the metabolome may be more useful than monitoring the transcriptome or proteome because the metabolic composition of a cell or organism provides its actual biochemical condition.

Infectious diseases and metabolic disorders may alter the metabolism of a living cell or organism. In other words, the metabolism of a healthy cell may be different than the metabolism of an unhealthy cell of the same type. Metabolism generally refers to the chemical processes of a living cell or organism that support and maintain life. The products of these chemical processes may be generally referred to as metabolites. The metabolism and/or metabolites of a living cell or organism may change depending on its biological state, developmental stage, history, and/or environment. Viruses, for example, may alter the metabolism of an infected cell. The metabolisms and/or metabolites of a virally infected cell and an uninfected cell of the same type may be different. The identification and analysis of metabolites may facilitate the detection, prevention, and/or treatment of infectious diseases and metabolic disorders.

Mass spectrometry is an analytical technique that has been successfully used in chemistry, biology, and other fields for qualitative and quantitative analysis. The identification and analysis of metabolites by conventional methods of mass spectrometry may be problematic. For example, matrix-assisted laser desorption ionization (MALDI) and electrospray ionization (ESI), may suffer from time consuming and complex sample preparation, and in situ analysis of a sample may be difficult. Atmospheric pressure mass spectrometry methods, such as direct analysis in real time (DART), desorption electrospray ionization (DESI), atmospheric pressure infrared matrix-assisted laser desorption ionization (AP IR-MALDI), desorption atmospheric pressure chemical ionization (DAPCI), matrix-assisted laser desorption electrospray ionization (MALDESI), and electrospray laser desorption ionization (ELDI), may suffer from other limitations, including, but not limited to, complex and time consuming separation techniques, a narrower range of samples that may be analyzed, higher detection limits, sensitivity to surface properties, sampling errors, and/or lack of imaging and quantization capabilities. Mass spectrometry may be combined with separation techniques, such as gas chromatography, high performance liquid chromatography and capillary electrophoresis, however, these techniques may increase the analysis time and/or cost.

Therefore, more efficient and/or cost-effective mass spectrometers and methods of making and using the same are desirable.

### SUMMARY

In certain embodiments, more efficient and/or cost-effective mass spectrometers and methods of making and using the same are described.

According to certain embodiments, a method of mass spectrometry may generally comprise subjecting a sample comprising at least one indicator to laser ablation electrospray ionization mass spectrometry; determining a relative intensity of the indicator; and comparing the relative intensity of the indicator to a standard indicator intensity.

### DESCRIPTION OF THE DRAWING FIGURES

The various embodiments described herein may be better understood by considering the following description in conjunction with the accompanying drawing figures.

FIGS. 1-2 include illustrations of LAESI ion sources for mass spectrometers in accordance with various embodiments described herein.

FIGS. 3A and 3B include photographs illustrating an ablation plume (LA) and an electrospray plume (ES) in accordance with various embodiments described herein.

FIGS. 4-5 include flow diagrams illustrating methods of mass spectrometry in accordance with various embodiments described herein.

FIGS. 6-7 include tables listing representative data from methods of mass spectrometry in accordance with various embodiments described herein.

FIGS. 8A and 8B include representative mass spectra in accordance with various embodiments described herein.

FIGS. 9A-D include representative mass spectra in accordance with various embodiments described herein.

FIGS. 10A-C include representative mass spectra in accordance with various embodiments described herein.

FIG. 11 includes a flow diagram illustrating the metabolic pathways involving putrescine, spermidine, and spermine.

FIG. 12 includes a chart comparing CEM and C81 cells in accordance with various embodiments described herein.

FIGS. 13A and 13B include representative mass spectra in accordance with various embodiments described herein.

FIGS. 14A-C include charts comparing CEM and C81 cells in accordance with various embodiments described herein.

FIGS. 15-16 include tables listing representative data from methods of mass spectrometry in accordance with various embodiments described herein.

FIG. 17 includes a flow diagram illustrating creatine and polyamine biosynthesis pathways in T lymphocytes.

FIG. 18 includes representative mass spectra in accordance with various embodiments described herein.

FIG. 19 includes a flow diagram illustrating a lipid metabolism pathway in T lymphocytes.

FIGS. 20A and 20B include a flow diagram illustrating metabolites detected in accordance with various embodiments described herein.

### DESCRIPTION OF CERTAIN EMBODIMENTS

### A. Definitions

As generally used herein, the terms “consisting essentially of and “consisting of” are embodied in the term “comprising”.

As generally used herein, the articles “one”, “a”, “an” and “the” refer to “at least one” or “one or more”, unless otherwise indicated.

As generally used herein, the terms “including” and “having” mean “comprising”.

As used herein, the terms “LAESI” and “LAESI-MS” refer to laser ablation electrospray ionization mass spectrometry.

As used herein, the term “infection” refers to the invasion by, multiplication and/or presence of a virus or bacteria in a cell, tissue, organ and/or organism.

As used herein, the term “metabolic disorder” refers to any pathological condition in a cell, tissue, organ and/or organism resulting from an alteration in an organism's metabolism.

As used herein, the term “metabolome” refers to the total or partial set of metabolites in a cell, tissue, organ and/or organism at a specific time. An infection and/or metabolic disorder may cause certain metabolites to be upregulated or down-regulated.

As used herein, the term “pattern” refers to a set of metabolites and their intensities measured by a diagnostic method. The set of metabolites may be the total or partial set of metabolites in a cell, tissue, organ and/or organism at a specific time

As generally used herein, the terms “about” and “approximately” refer to an acceptable degree of error for the quantity measured, given the nature or precision of the measurements. Typical exemplary degrees of error may be within 20%, 10%, or 5% of a given value or range of values. Alternatively, and particularly in biological systems, the terms “about” and “approximately” refer to values within an order of magnitude, potentially within 5-fold or 2-fold of a given value.

All numerical quantities stated herein are approximate unless stated otherwise; meaning that the term “about” may be inferred when not expressly stated. The numerical quantities disclosed herein are to be understood as not being strictly limited to the exact numerical values recited. Instead, unless stated otherwise, each numerical value is intended to mean both the recited value and a functionally equivalent range surrounding that value. At the very least, and not as an attempt to limit the application of the doctrine of equivalents to the scope of the claims, each numerical parameter should at least be construed in light of the number of reported significant digits and by applying ordinary rounding techniques. Notwithstanding the approximations of numerical quantities stated herein, the numerical quantities described in specific examples of actual measured values are reported as precisely as possible.

All numerical ranges stated herein include all sub-ranges subsumed therein. For example, a range of “1 to 10” is intended to include all sub-ranges between and including the recited minimum value of 1 and the recited maximum value of 10. Any maximum numerical limitation recited herein is intended to include all lower numerical limitations. Any minimum numerical limitation recited herein is intended to include all higher numerical limitations.

In the following description, certain details are set forth in order to provide a better understanding of various embodiments of mass spectrometers and methods for making and using the same. However, one skilled in the art will understand that the embodiments described herein may be practiced without these details. In other instances, well-known structures and methods associated with mass spectrometers and methods of mass spectrometry may not be shown or described in detail to avoid unnecessarily obscuring descriptions of the embodiments of this disclosure.

This disclosure describes various features, aspects, and advantages of various embodiments of mass spectrometers and methods for making and using the same. It is understood, however, that this disclosure embraces numerous alternative embodiments that may be accomplished by combining any of the various features, aspects, and advantages of the various embodiments described herein in any combination or sub-combination that one of ordinary skill in the art may find useful.

### B. Overview

Infection of a cell or organism causes extensive changes at the gene, protein, and metabolite levels. These changes are usually followed by gene-expression profiling and proteomic analysis. Viruses, for example, rely on the metabolic network of their cellular hosts for survival and replication. Exploring the metabolic consequences of a viral infection may provide insight into the causes and/or treatment of viral infection. The insight gained by such studies may depend on the target sample, treatment procedures, and detection techniques used. Conventionally, biofluids, such as blood and urine, have been used to follow metabolic changes after infection, but in many cases they may complicate the analysis due to the pooling of changes in different cell types and the variations between individuals. Ultimately, direct analysis of a sample is a more straightforward way to understand the actual disease-associated metabolic changes in and at the site of an infection. In such cases, a direct detection technique may offer key advantages.

Metabolites are small molecules of diverse physico-chemical properties with greatly different abundance levels that make their identification and analysis challenging. Typically, optical spectrometry, such as Fourier transform infrared spectrometry, nuclear magnetic resonance (NMR), and mass spectrometric techniques in combination with separation techniques, such as gas chromatography, high performance liquid chromatography (HPLC) and capillary electrophoresis, have been used for metabolomic studies. Mass spectrometry (MS) is a versatile technique that, combined with chromatographic separations, may provide qualitative and quantitative analyses of complex samples with high selectivity and sensitivity, as well as a broad dynamic range. Conventional mass spectrometric methods, however, are time consuming and involve extensive sample preparation. The application of direct sampling methods, such as flow injection electrospray ionization (ESI), may avoid chromatographic separation, but not the extensive sample preparation that may affect sample integrity and, in some cases, may lead to metabolite degradation. Therefore, these techniques generally restrict the choice of samples and discourage their in situ analysis.

Some of these problems may be mitigated by the use of atmospheric pressure ion sources. Recent advances in atmospheric pressure ion sources, such as direct analysis in real time (DART), desorption electrospray ionization (DESI), atmospheric pressure infrared matrix-assisted laser desorption ionization (AP IR-MALDI), and laser ablation electrospray ionization (LAESI) may enable direct analysis of cell and tissue samples without extensive sample preparation. Analysis of cells, cell cultures, and cell extracts using DART, DESI, and MALDI techniques may have their own limitations, such as coverage of analytes, sampling of the surface only, and quantitation restrictions. According to certain embodiments, LAESI-MS may provide for in situ cell and tissue analysis, and may sample the entire volume of the cells for metabolites and lipid components with tissue imaging and quantitation capabilities.

Previous studies of viral infection indicate that glycolysis, the citrate (TCA) cycle, pyrimidine nucleotide biosynthesis, and lipid metabolism are the main metabolic changes generally involved with viral infection. The metabolic intermediates of these pathways may increase in response to the infection, e.g., faster uptake of glucose and glutamine, greater accumulation of citrate, and increasing excretion of lactate and glutamate in infected cells. The efflux from infected cells may be enriched in metabolites related to nucleotide and fatty acid biosynthesis. The fatty acid biosynthesis in infected cells may be considered an antiviral response. Previous studies of HIV infected cells indicate a reduction of glutathione levels, changes in lipid metabolism, and polyamine pools (putrescine, spermidine and spermine). However, these studies were carried out using conventional MS-based techniques involving both sample extraction and chromatographic separation. According to certain embodiments, LAESI-MS provides rapid and direct identification of metabolic changes in HTLV1 infected T lymphocytes and reduces and/or eliminates the need for extensive sample preparation.

Human T cell leukemia virus type I (HTLV1), a member of the delta-retroviridae subfamily, was the first human pathogenic retrovirus discovered. HTLV1 may contribute to cancer development. Infection with HTLV1 may result in the development of adult T-cell leukemia (ATL), a CD4⁺T lymphoproliferative malignancy. Estimates of worldwide HTLV1 infections may be between 15 million to 25 million individuals. However, infected individuals develop ATL after a long latent period and at a 3-5% incidence rate. Infection with HTLV1 may result in HTLV1-associated myelopathy/tropical spastic paraparesis (HAM/TSP) and several inflammatory diseases, including polymyositis, uveitis, and lymphocyte alveolitis. The development of ATL from HTLV1 infection may be a multi-hit occurrence with initial transformation due to the viral protein Tax. Recent studies have indicated that the use of novel treatments, including monoclonal antibodies against the interleukin-2 receptor (IL-2R) and the combination therapy of interferon-alpha (IFN-α) and zidovudine (AZT), to be effective, but only in a small percentage of ATL patients. Therefore, new therapies for the treatment of ATL, and in particular, HTLV1 infection, are desirable.

According to certain embodiments, mass spectrometers and methods of mass spectrometry for identifying and analyzing changes in the metabolic profiles of a cell, tissue, and/or organism after infection and/or development of a metabolic disorder. The metabolic changes in virally infected cells and tissues may be monitored using high throughput metabolomic and complementary transcriptomic and proteomic technologies, such as LAESI-MS, as described herein. In certain embodiments, LAESI-MS may be used to identify and analyze changes in the metabolic profiles of a cell, tissue, and/or organism after HTLV1 infection, HTLV3 infection, Tax1 expression, and Tax3 expression as well as comparing the metabolic profiles of cells and tissues transfected with either HTLV3 molecular clone or Tax3 and HTLV1 transformed cells. Understanding the role of Tax in destabilizing key regulators, such as proteins, in metabolism and cell cycle control, may help identify molecular markers that contribute to ATL development and define new therapeutic strategies. Certain embodiments comprise in situ metabolite profiling of infected cells. Certain embodiments comprise facilitating the identification of virus-induced perturbations in the biochemical processes of a host cell.

Certain embodiments of the mass spectrometers and methods of making and using the same described herein may provide certain advantages over other approaches of mass spectrometric analysis. The advantages may include, but are not limited to, in situ analysis, simultaneous detection of multiple samples, independent optimization of ablation conditions and ionization conditions, a wider dynamic range of samples that may be used, operation under ambient conditions, simpler sample preparation, minimal sample manipulation, minimal sample degradation, improved sampling time, positional sensitivity, improved sensitivity to surface properties, and/or improved detection limits.

### C. Laser Ablation Electrospray Ionization Mass Spectrometry

According to certain embodiments, a mass spectrometer for laser ablation electrospray ionization mass spectrometry may generally comprise a laser system, an electrospray apparatus, and a mass spectrometer. The laser system may comprise a laser and a focusing system comprising fiber optics, coupling lenses, and/or focusing lenses, and an x-y-z translation stage having a sample mount. The laser may be selected from the group consisting of an Er:YAG laser, an Nd:YAG laser driven optical parametric oscillator and a free electron laser. The electrospray apparatus may comprise an electrospray ionization emitter having a power supply and a syringe pump. The mass spectrometric ion source may comprise a solid state camera. The mass spectrometric ion source may comprise a shroud to enclose the sample, the sample holder, and/or the electrospray emitter. The translation stage and the sample environment may be temperature controlled and/or atmosphere controlled. This is to maintain sample integrity and to avoid condensation of moisture from the environment. The atmosphere may comprise ambient atmosphere. The temperature may ranges from −10° C. to 60° C. The relative humidity may range from 10% to 90%.

In certain embodiments, the atmosphere and/or the electrospray solution may comprise a reactive component to facilitate the ionization and/or fragmentation of certain constituents of the sample. For example, the electrospray solution may comprise Li₂SO₄ to facilitate the structural identification of lipids by inducing structure specific fragmentation in collision induced dissociation experiments. Examples of reactive gases include, but are not limited to, ammonia, SO₂, and NO₂.

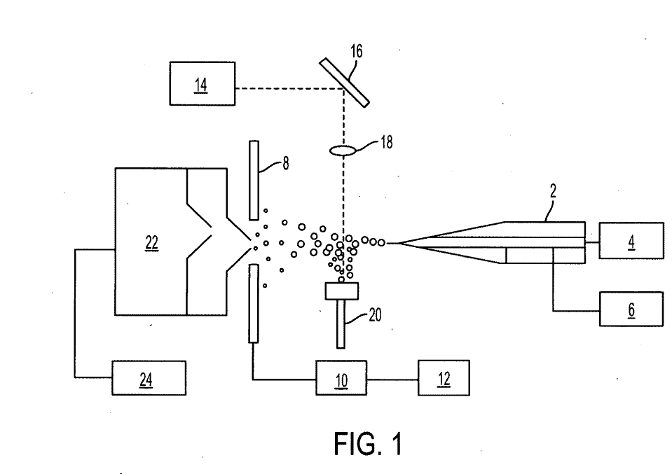

Referring to FIG. 1, in certain embodiments, a mass spectrometer comprising a LAESI ion source may generally comprise an electrospray capillary 2, an optional liquid supply with pump 4, a high voltage power supply 6, a counter electrode 8, an oscilloscope 10, a recording device 12, e.g., personal computer, a laser 14, such as an Er:YAG laser or Nd:YAG laser driven optical parametric oscillator, a beam steering device 16, e.g., a minor, a focusing device 18, e.g., lens or sharpened optical fiber, a sample holder with x-y-z positioning stage 20, a mass spectrometer 22, and a recording device 24, e.g., a personal computer.

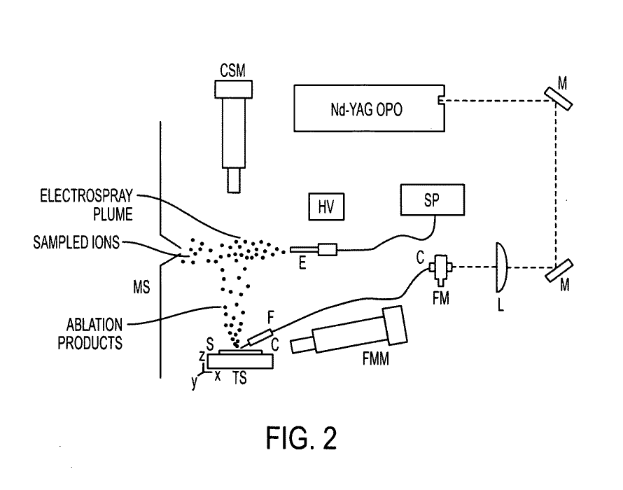

Referring to FIG. 2, in certain embodiments, a mass spectrometer comprising a LAESI ion source may generally comprise an electrospray capillary (E), an optional a liquid supply with pump (SP), a high voltage power supply (HV), a laser, such as an Er:YAG laser or Nd:YAG laser driven optical parametric oscillator, beam steering devices, e.g., a mirror (M), a focusing device, e.g., lens or sharpened optical fiber, a sample holder with x-y-z positioning stage (TS), a long-distance video microscope (FMM), a second video microscope (CSM), and a mass spectrometer (MS). In certain embodiments, a mass spectrometer comprising a LAESI ion source may comprise an etched optical fiber tip (F) to generate mid-IR ablation products that may be intercepted by the electrospray plume and post-ionized to form ions sampled by the mass spectrometer (MS), a long-distance video microscope (fiber monitor, FMM) to maintain a constant distance between the fiber tip and the sample surface (S), a sample placed on a three-axis translation stage (TS), and a second video microscope (cell spotting microscope, CSM) to target the sample. The electrospray may be produced by applying high voltage (HV) to the capillary emitter (E) and by maintaining a constant solution flow rate by a syringe pump (SP). Pulses from the laser may be coupled to the optical fiber, adjusted by a fiber chuck (C) and a five-axis fiber mount (FM), using two Au-coated mirrors (M) and a CaF₂ lens (L). The LAESI-MS device may comprise a recording device, e.g., a personal computer. The mass spectrometer comprising a LAESI ion source may be configured for single cell analysis. The LAESI-MS method for a single cell may comprise using micromanipulators and reducing the laser spot size from 5 μm to 200 μm.

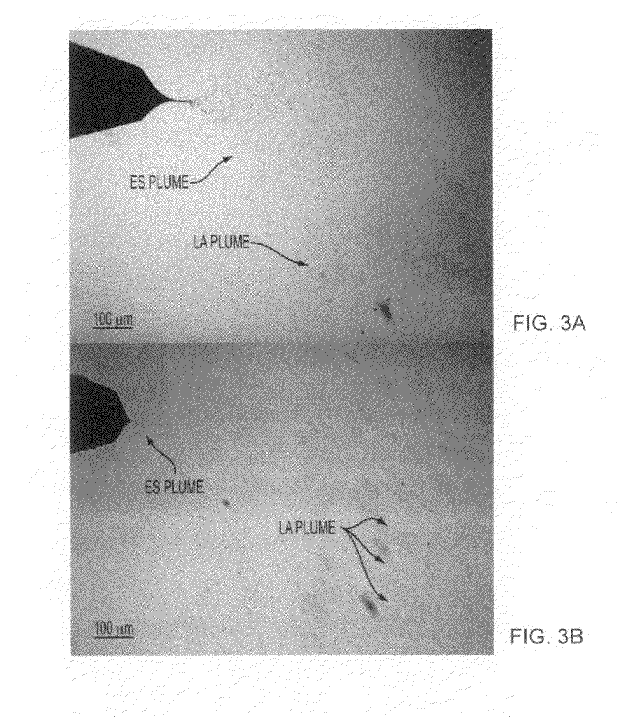

In certain embodiments, an ablation plume (LA) may intersect an electrospray plume (ES). Referring to FIGS. 3A and 3B, the electrosprayed droplets travel downstream from the emitter (from left to right). The electrosprayed droplets are intercepted by particulates traveling upward from the ablation plume. The ablation plume may comprise 1 μm to 3 μm particles. Without wishing to be bound to any particular theory, at the intersection of the two plumes, some of the ablated particulates may fuse with the electrospray droplets to form charged droplets that contain some of the ablated material, and ultimately produce ions in an ESI process. The electrospray emitter may be operated in a pulsating spraying regime and/or a cone-jet regime. As shown in FIG. 3A, the pulsating spraying regime may offer a lower duty cycle and produce larger electrospray droplets resulting in lower ionization efficiency and LAESI signal. As shown in FIG. 3B, the cone-jet regime may produce smaller electrospray droplets resulting in higher ionization efficiency and LAESI signal.

In certain embodiments, the laser may comprise an infrared laser. The infrared laser may operate at a wavelength from 2600 nm to 3450 nm, such as 2800 nm to 3200 nm, and 2930 nm to 2950 nm. The laser may comprise a mid-infrared pulsed laser operating at a wavelength from 2600 nm to 3450 nm, a repetition rate from 1 Hz to 100 Hz, and a pulse width from 0.5 ns to 50 ns. In at least one embodiment, the laser may comprise a diode pumped Nd:YAG laser-driven optical parametric oscillator (OPO) (Opolette 100, Opotek, Carlsbad, Calif.) operating at 2940 nm, 100 Hz repetition rate, and 5 ns pulse width. The optical fiber may comprise a germanium oxide (GeO₂)-based optical fiber (450 μm core diameter, HP Fiber, Infrared Fiber Systems, Inc., Silver Spring, Md.) with its tip etched to a radius of curvature from 1 μm to 50 μm, such as 5 μm to 25 μm, and 10 μm to 15 μm. In at least one embodiment, the radius of curvature may be 15 μm. The optical fiber may deliver the laser pulse to the sample. The energy of a laser pulse before coupling into the optical fiber may be from 0.1 mJ to 6 mJ, thus the pulse-to-pulse energy stability generally corresponds to 2% to 10%. In at least one embodiment, the energy of a laser pulse before coupling into the optical fiber may be 554±26 μJ, thus the pulse-to-pulse energy stability generally corresponds to 5%. The laser system may be operated at 100 Hz from 0.01 seconds to 20 seconds to ablate a sample. In at least one embodiment, laser system may be operated at 100 Hz for 1 second to ablate a sample. In certain embodiments, 1 to 100 laser pulses may be delivered to a sample for analysis.

In certain embodiments, the electrospray source may comprise a low noise syringe pump (Physio 22, Harvard Apparatus, Holliston, Mass.) to supply the electrospray solution to a tapered stainless steel emitter (inner diameter 50 μm, MT320-50-5-5, New Objective, Woburn, Mass.). The low noise syringe pump may supply the electrospray solution at a rate from 10 mL/min to 10 μL/min. In at least one embodiment, the low noise syringe pump may supply the electrospray solution at 200 mL/min. The tapered stainless steel emitter may have an outside diameter from 100 μm to 500 μm and an insider diameter from 10 μm to 200 μm. The power supply may comprise a regulated power supply (PS350, Stanford Research Systems, Sunnyvale, Calif.), to provide a stable high voltage from 2.5 to 5 kV to the electrospray emitter. The power supply may be mounted on a manual translation stage to optimize the LAESI signal by adjusting the relative position of the sample, electrospray emitter, and/or inlet orifice of the mass spectrometer. The electrospray solution may comprise at least one of 50% methanol with 0.1% (v/v) acetic acid, 50% methanol with 0.1% (v/v) formic acid, 50% methanol with 0.1% (v/v) trifluoroacetic acid, 50% methanol with 0.1% (v/v) ammonium acetate. The electrospray solution may be applied at an angle from 0° to 90°, such as 30°, 45°, and 60°, into the ablation plume. The angle may be adjusted from 0° to 90° to optimize ion production. In at least one embodiment, the electrospray solution may be applied at a right angle (90°) into the ablation plume.

According to certain embodiments, the mass spectrometer orifice may be on the same or a different axis as the electrospray emitter of the LAESI ion source. The angle between the mass spectrometer orifice and electrospray emitter of the LAESI ion source may be from 0° to 90°, such as 30°, 45°, and 60°. The distance from the mass spectrometer orifice to the electrospray emitter tip may be from 1 mm to 20 mm, such as 5 mm to 15 mm. In at least one embodiment, the distance from the mass spectrometer orifice to the electrospray emitter tip may be 12 mm. The sample may be placed onto a pre-cleaned microscope glass slide (catalog no. 125496, Fisher Scientific, Pittsburgh, Pa.). The sample may be placed onto a stepper motor-driven three axis precision flexure stage (NanoMax TS, Thorlabs, Newton, N.J.). The sample may be 1 mm to 30 mm below the spray axis, such as 5 mm to 25 mm, and 10 mm to 20 mm. In at least one embodiment, the sample may be 15 mm below the spray axis. In one experiment, no ions were detected by the mass spectrometer when the ESI was off, indicating that no ions directly induced by the laser were collected. This observation may result from the large (>15 mm) distance between the orifice of the mass spectrometer and the ablated sample.

The positive ions produced by the LAESI ion source may be analyzed by a mass spectrometer. The mass spectrometer may comprise an orthogonal acceleration time-of-flight mass spectrometer (QTOF Premier, Waters Co., MA). The orifice of the mass spectrometer may have an inner diameter from 100 μm to 500 μm, such as 225 μm to 375 μm. In at least one embodiment, the orifice of the mass spectrometer may have an inner diameter from 100 μm to 200 μm, such as 127 μm. The orifice of the mass spectrometer may be extended by a straight or curved extension tube having a similar inner diameter as the orifice of the mass spectrometer and a length from 20 mm to 500 mm. The interface block temperature may be from ambient temperature to 150° C., such as 23° C. to 90° C., and 60° C. to 80° C. In at least one embodiment, the interface block temperature may be 80° C. The potential may be from −100 V to 100 V, such as −70 V to 70 V. In at least one embodiment, the potential may be −70 V. Tandem mass spectra may be obtained by collision activated dissociation (CAD) with a collision gas, such as argon, helium or nitrogen, at a collision cell pressure from 10⁻⁶ mbar to 10⁻² mbar, and with collision energies from 10 eV to 200 eV. In at least one embodiment, the collision gas may be argon, the collision cell pressure may be 4×10⁻³ mbar, and the collision energies may be from 10 eV to 25 eV.

In certain embodiments, the laser beam may be steered by gold-coated minors (PF10-03-M01, Thorlabs, Newton, N.J.) and coupled into the cleaved end of the optical fiber by a plano-convex calcium fluoride lens (Infrared Optical Products, Farmingdale, N.Y.) having a focal length from 2 mm to 100 mm, such as 25 mm to 75 mm, and 40 mm to 60 mm. In at least one embodiment, the focal length may be 50 mm. The optical fiber may be held by a bare fiber chuck (BFC300, Siskiyou Corporation, Grants Pass, Oreg.). The optical fiber may be positioned by a five-axis translator (BFT-5, Siskiyou Corporation, Grants Pass, Oreg.).

In certain embodiments, the optical fiber may comprise a GeO₂-based glass fiber, a fluoride glass fiber, and a chalcogenide fiber. The optical fiber may have a high laser-damage threshold due to its high glass transition temperature. The Hytrel and polyimide coatings may be stripped off both ends of the fiber by the application of 1-methyl-2-pyrrolidinone (at 130° C. to 150° C. for 1 min). After stripping off the Hytrel and the polyimide coatings, the fiber ends may be cleaved with a Sapphire blade (KITCO Fiber Optics, Virginia Beach, Va.) by scoring and gently snapping them. Chemical etching of the GeO₂-based glass fiber tip may be achieved by dipping one of the cleaved fiber ends 0.5 mm deep into 24° C. 1% HNO₃ solution in a wide beaker to provide a low meniscus curvature. The meniscus formed at the fiber end may gradually etch the 450 μm diameter core into a sharp tip having a radius of curvature (R) of 15 μm. Prior to use, the etched tips may be washed with deionized water. In certain embodiments, no visible change of the fiber tip may be observed after performing the LAESI technique which may indicate the absence of damage or contamination.

In certain embodiments, the etched end of the fiber may be attached to a micromanipulator (MN-151, Narishige, Tokyo, Japan) to move the etched end of the fiber closer to the sample. The distance from the etched end of the fiber and the sample may be from contact (0 μm) to 50 μm. In at least one embodiment, the coordinate system may be aligned so that the x-y plane coincides with the sample and the x-axis is parallel with the emitter, the optical fiber is positioned at an azimuth angle from 20° to 160° and a zenith angle from 20° to 70°. In at least one embodiment, the azimuth angle may be 135° and the zenith angle may be 45°. The zenith angle of 45° may provide an acceptable trade-off between the shape of the ablation mark and signal intensity reduction by blocking the expanding plume. A thin sample material deposit may be observed on the fiber tip after ablation. In these cases, the fiber may be retracted from the surface and elevated laser pulse energy may be used to clean the tip. In at least one embodiment, the distance between the fiber tip and the sample surface (h) may be 2 R. This may result in an ablation mark with an average diameter of 2.5 R. In at least one embodiment, the distance between the fiber tip and the sample surface may be 30 μM, resulting in an ablation mark with an average diameter of 37.5 μm. Microscope images of the ablation marks may be obtained by an upright microscope (BX 51, Olympus America Inc., Center Valley, Pa.) in either reflected or transmitted mode and by an inverted microscope.

In certain embodiments, the mass spectrometer ion source may comprise a visualization system. The distance between the fiber tip and sample surface may be monitored by a long distance video microscope (InFocus Model KC, Infinity, Boulder Colo.) with a 5× infinity corrected objective lens (M Plan Apo 5×, Mitutoyo Co., Kanagawa, Japan), and the image may be captured by a CCD camera (Marlin F131, Allied Vision Technologies, Stadtroda, Germany). With the environmental vibration in the low micrometer range, an approximate distance from 30 μm to 40 μm may be maintained between the tip and the sample. A similar video microscope system may be used at a right angle to the sample surface to align the fiber tip over the location of interest in the sample for ablation. The visualization system may comprise a 7× precision zoom optic (Edmund Optics, Barrington, N.J.), fitted with a 10× infinity-corrected long working distance objective lens (M Plan Apo 10×, Mitutoyo Co., Kanagawa, Japan) and a CCD camera (Marlin F131, Allied Vision Technologies, Stadtroda, Germany).

In certain embodiments, the sample may comprise any sample that comprises water, including, but not limited to, cells, tissues, organs, biofilms, aqueous solutions, organic materials, inorganic materials, synthetic materials, biomedical samples, forensic samples, biological warfare agents, and wetted surfaces. For example, the sample may be selected from the group consisting of biomolecules (such as metabolites, lipids, nucleic acids, proteins, peptides, and carbohydrates), organic and inorganic molecules (such as pharmaceuticals, polymers, dendrimers and other macromolecules), and mixtures thereof. The sample may comprise a biofilm comprising an aggregate of microorganisms in which cells adhere to each other and/or to a surface. The sample may comprise a single cell, cells, small cell populations, cell lines, and tissues. The single cell may have a smallest dimension less than 100 micrometers, such as less than 50 μm, less than 25 μm, and/or less than 10 μm. The single cell may have a smallest dimension from 1 μm to 100 μm, such as from 5 μm to 50 μm, and from 10 μm to 25 μm. In at least one embodiment, the single cell may have a smallest dimension from 1 μm to 10 μm. The small cell population may comprise 10 to 1 million cells, such as 50 cells to 100,000 cells, and 100 cells to 1,000 cells. The sample may comprise a cell infected with at least one of a virus and a bacterium. The sample may comprise a virally infected living cell and/or tissue. The sample may comprise a cell having a metabolic disorder.

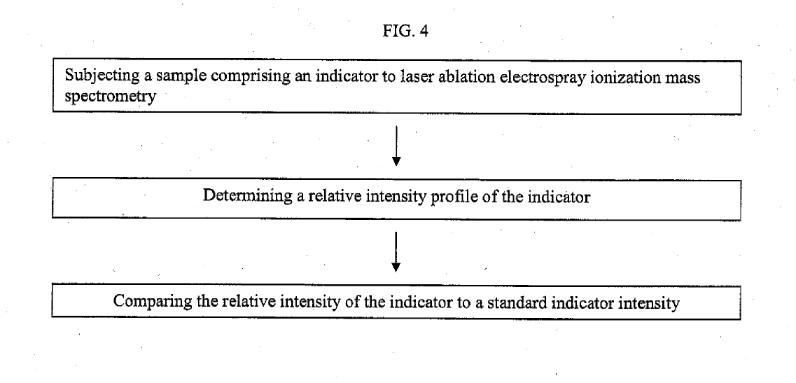

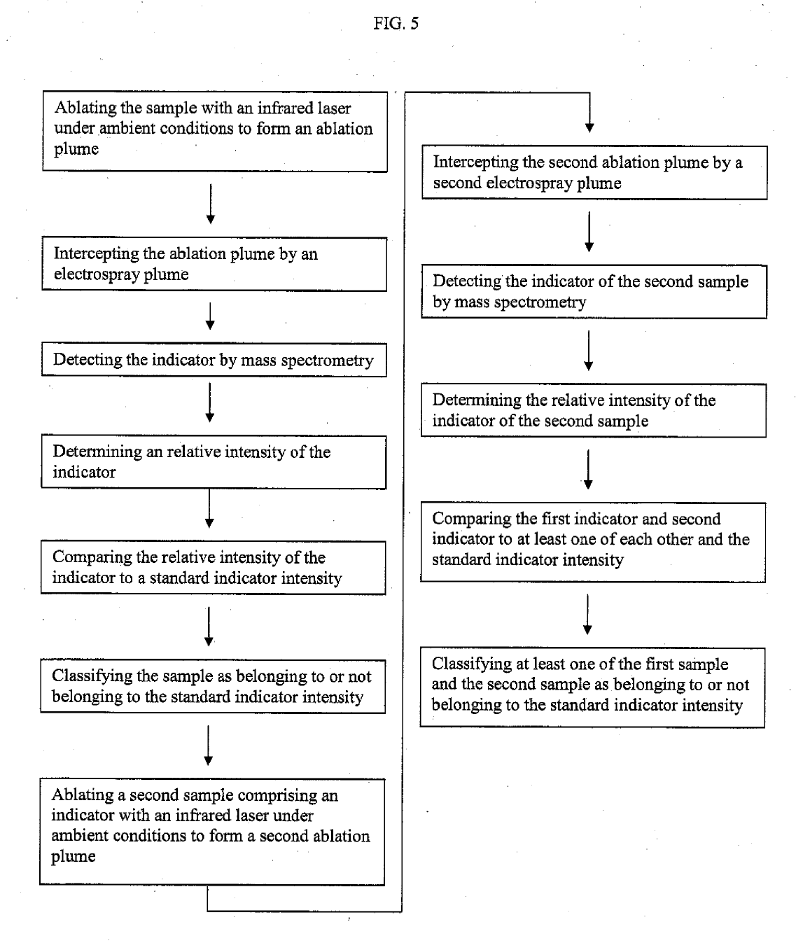

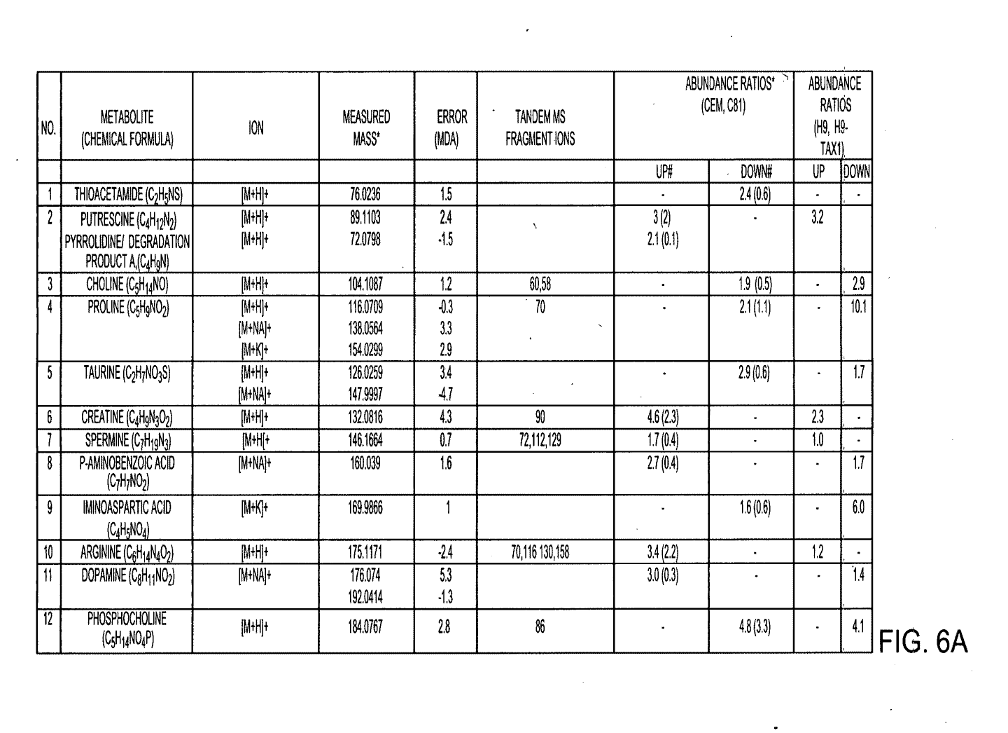

Referring to FIG. 4, according to certain embodiments, a method of mass spectrometry may generally comprise subjecting a sample comprising an indicator to laser ablation electrospray ionization mass spectrometry; determining a relative intensity of the indicator; and comparing the relative intensity of the indicator to a standard indicator intensity. Subjecting the sample to laser ablation electrospray ionization mass spectrometry may comprise ablating the sample with an infrared laser under ambient conditions to form an ablation plume; intercepting the ablation plume by an electrospray plume; and detecting the indicator by mass spectrometry. In certain embodiments, subjecting the sample to laser ablation electrospray ionization mass spectrometry may exclude pretreating the sample with a matrix material. In certain embodiments, determining the relative intensity of the indicator may comprise determining the relative intensity of an indicator comprising multiple charge states. The standard indicator intensity may comprise an internal reference and/or an external reference.

According to certain embodiments, the indicator may comprise a biomolecule, an organic molecule, a molecular complex and/or a xenobiotic. The indicator may be selected from the group consisting of metabolites, lipids, lipid precursors, lipid components, nucleic acids, proteins, peptides, carbohydrates, and combinations thereof. The indicator may comprise intracellular metabolites from a single cell. The indicator may comprise a biomarker. The biomarker may be related to a metabolic change. The biomarker may be related to a disease state. The disease state may be associated with a viral infection, a bacterial infection, and/or a metabolic disorder.

Viruses may include, but are not limited to, human immunodeficiency virus 1 (HIV-1), human immunodeficiency virus 2 (HIV-2), human T-lymphotropic virus type 1 (HTLV1), human T-lymphotropic virus type 2 (HTLV2), human T-lymphotropic virus type 3 (HTLV3), human papillomavirus (HPV), paramyxoviruses, hepatitis A, hepatitis B, hepatitis C, viral encephalitis, herpes virus, influenza, and/or severe acute respiratory syndrome (SARS). Bacteria may include, but are not limited to, Bacillus anthracis (the causative agent of anthrax), Yersinia pestis (the causative agent of bubonic plague), Mycobacterium tuberculosis (the causative agent of tuberculosis) and Vibrio cholera (the causative agent of cholera). The metabolic disorder may comprise diabetes, muscular dystrophy, phenylketonuria (PKU), Tay Sachs disease, leukodystrophies, lysosomal disorders, Wilson's disease, Lesch-Nyhan syndrome, urea cycle disorder, amyloidosis and/or lipid storage diseases. In certain embodiments, the disease state may comprise human immunodeficiency virus (HIV), human T-lymphotropic virus type 1 (HTLV1), and/or human T-lymphotropic virus type 3 (HTLV3) as well as Tax1 and/or Tax3 expressing cells, and the indicator may comprise glutathione, spermine, spermidine, putrescine, arginine, creatine, choline, phosphocholine, glycerophosphocholine, glycerophosphocholine lipids, ATP, ADP, AMP, cAMP, dopamine, dopamine metabolites, and/or any combination thereof. In certain embodiments, the indicator may be related to a metabolic change caused by exposure to a toxin. For example, the activity of an enzyme, butyrylcholinesterase, in blood may be used as a biomarker for nerve agent exposure.

According to certain embodiments, the method of mass spectrometry may comprise classifying the sample as belonging to or not belonging to the standard indicator intensity. A sample not belonging to the standard indicator intensity may indicate that the sample comprises a metabolic change. The metabolic change may be associated with a viral infection, a bacterial infection, and/or a metabolic disorder. A sample not belonging to the standard indicator intensity may indicate that the sample is predicted to comprise a disease state. The disease state may be associated with a viral infection, a bacterial infection, and/or a metabolic disorder.

In certain embodiments, the indicator may comprise a plurality of indicators. The method of mass spectrometry may comprise determining the relative intensity of each indicator to form a sample metabolite pattern and comparing the sample metabolite pattern to a standard metabolite pattern comprising the standard indicator intensity of each of the plurality of indicators. The method of mass spectrometry may comprise classifying the sample as belonging to or not belonging to the standard metabolite pattern. A sample not belonging to the standard metabolite pattern may indicate that the sample comprises a metabolic change. A sample not belonging to the standard metabolite pattern may indicate that the sample is predicted to comprise a disease state.

Referring to FIG. 5, according to certain embodiments, an in situ method of determining a metabolic state of a sample comprising an indicator may generally comprise ablating the sample with an infrared laser under ambient conditions to form an ablation plume; intercepting the ablation plume by an electrospray plume; detecting the indicator by mass spectrometry; determining a relative intensity of the indicator; comparing the relative intensity of the indicator to a standard indicator intensity; and classifying the sample as belonging to or not belonging to the standard indicator intensity. A sample not belonging to the standard indicator intensity may indicate that the sample comprises a metabolic change. A sample not belonging to the standard indicator intensity may indicate that the sample is predicted to comprise a disease state. The metabolic change may be associated with a viral infection, a bacterial infection, and/or a metabolic disorder.

In certain embodiments, determining the relative intensity of the indicator may comprise determining the relative intensity of an indicator comprising multiple charge states. The indicator may comprise a plurality of indicators. The method of mass spectrometry may comprise determining the relative intensity of each indicator to form a sample metabolite pattern and comparing the sample metabolite pattern to a standard metabolite pattern comprising the standard indicator intensity of each of the plurality of indicators. The method of mass spectrometry may comprise classifying the sample as belonging to or not belonging to the standard metabolite pattern. A sample not belonging to the standard metabolite pattern may indicate that the sample comprises a metabolic change. A sample not belonging to the standard metabolite pattern may indicate that the sample is predicted to comprise a disease state. The standard indicator intensity may comprise an internal reference and/or an external reference.

In certain embodiments, the metabolic state may be selected from the group consisting of developmental stages, for example, the stages of a cell cycle, environments, nutritional supplies, taxonomic units, genetic units, infected and uninfected states, diseased and healthy states, and different stages of pathogenicity.

According to certain embodiments, the method of determining a metabolic state may comprise ablating a second sample comprising the indicator with an infrared laser under ambient conditions to form a second ablation plume; intercepting the second ablation plume by a second electrospray plume; detecting the indicator of the second sample by mass spectrometry; determining the profile of the indicator of the second sample; comparing the first indicator and second indicator to each other and/or the standard indicator intensity; and classifying at least one of the first sample and the second sample as belonging to or not belonging to the standard indicator intensity. A sample not belonging to the standard indicator intensity may indicate that the sample comprises a metabolic change. A sample not belonging to the standard indicator intensity may indicate that the sample is predicted to comprise a disease state. The metabolic change and/or disease state may be associated with a viral infection, a bacterial infection, and/or a metabolic disorder. In certain embodiments, comparing the first indicator and second indicator to each other may indicate that the sample comprises a metabolic change. In certain embodiments, comparing the first indicator and second indicator to each other may indicate that the sample is predicted to comprise a disease state.

In certain embodiments, the method of mass spectrometry may comprise determining temporal and/or spatial information from a sample. The second sample may comprise the first sample at a different time. For example, the first sample may comprise a cell and/or tissue and the second sample may comprise the same cell and/or tissue at a later time, such as after a predetermined period of time or at different stages in the cell cycle. The second sample may comprise a different portion of the first sample. For example, the first sample may comprise a first portion of a cell and/or tissue and the second sample may comprise another portion of the same cell and/or tissue.

According to certain embodiments, the metabolic changes in virally infected cells/tissues may be monitored using LAESI-MS. In certain embodiments, changes in metabolites and lipids may be directly detected from uninfected T lymphocytes, human T-lymphotropic virus type 1 (HTLV1) transformed cells, and human T-lymphotropic virus type 3 (HTLV3) transformed cells, and Tax1 and Tax3 expressing cell lines T lymphocytes. The mass spectra of uninfected and infected cells may be compared to identify any metabolic changes. Glycerophosphocholine (PC) lipid components may be dominant in the non-HTLV1 transformed cells and PC(O-32:1) and PC(O-34:1) plasmalogens may be displaced by PC(30:0) and PC(32:0) species in the HTLV1 transformed cells. In HTLV1 transformed cells choline, phosphocholine, spermine and glutathione, among others, may be downregulated, whereas creatine, dopamine, arginine and AMP may be upregulated. In certain embodiments, individual measurements on the T-cells may take a few seconds enabling high throughput studies using LAESI-MS. Analysis of different cell lines transfected with either the HTLV3 molecular clone or Tax3 may reveal metabolite changes that correlate to HTLV1 and HTLV3 infected cells, whereas others may be unique to HTLV1.

According to certain embodiments, the method of mass spectrometry may comprise identifying virus-induced perturbations in the biochemical processes of the host cell by high throughput in situ metabolite profiling of HTLV1 and HTLV3 transformed cells. This method may be used to better understand the molecular mechanisms of HTLV1 and HTLV3 infections, which in turn may result in drug development and/or new treatment strategies.

According to certain embodiments, the method of mass spectrometry may comprise analyzing cell-to-cell metabolic variations and/or analyzing cells at different stages of the cell cycle. In certain embodiments, a method of mass spectrometry may comprise identifying metabolic changes in HTLV1 and Tax1 transformed T lymphocytes and HTLV3 and Tax3 transformed kidney epithelial cells. The indicators may comprise glutathione, spermine, choline, phosphocholine, glycerophosphocholine, thioacetamide, proline, taurine, carbamoyl phosphate, methoxytyramine and 8-hydroxy guanosine, creatine, arginine, dopamine, homovanillic acid and AMP. The levels of glutathione, spermine, choline, phosphocholine, glycerophosphocholine, thioacetamide, proline, taurine, carbamoyl phosphate, methoxytyramine and 8-hydroxy guanosine may be downregulated in C81 cells. The levels of creatine, arginine, dopamine, homovanillic acid and AMP may be upregulated in infected cells.

In certain embodiments, the method of mass spectrometry may comprise comparing metabolic changes detected in H9-Tax1 cells and H9 cells as well as HUT102 cells and H9 cells. Some of the key metabolic changes detected in C81 cells were also observed in H9-Tax1 cells vs. H9 cells as well as HUT102 cells vs. H9 cells. Without wishing to be bound to any particular theory, these indicators participate in biochemical pathways, such as polyamine biosynthesis, creatine biosynthesis, AMP biosynthesis, dopamine metabolism, lipid metabolism, redox reactions etc. Comparing HTLV1 and HTLV3 transfected cells revealed that part of the metabolic response was similar, but there were several changes specific to HTLV1. For example, the changes in the levels of putrescine, taurine, arginine and AMP were consistent among HTLV1 transformed cells.

The various embodiments described herein may be better understood when read in conjunction with the following representative examples. The following examples are included for purposes of illustration and not limitation.

### D. Examples

The uninfected T lymphocyte cells (CEM and H9) and kidney epithelial cells (293T), HTLV1 infected cells (C81 and HUT102), and H9 cells stably transfected with Tax1 of HTLV1 (H9-Tax1) and 293T cells infected with HTLV3 (293T-HTLV3) and expressing Tax3 (293T-Tax3) were maintained in RPMI 1640 medium containing fetal bovine serum, L-glutamine (2 mM), penicillin (100 units/mL) and streptomycin (100 μg/mL). The medium solutions and buffers were procured from Quality Biological Inc. (Gaithersburg, Md.) and the solvents were HPLC grade available from Acros Organics (Geel, Belgium). The glacial acetic acid was procured from Fluka (Munich, Germany).

According to certain embodiments, the method of mass spectrometry was performed by a infrared laser system. An optical parametric oscillator (OPO) (Opolette 100, Opotek, Carlsbad, Calif.) converted the output of a 100 Hz repetition rate Nd:YAG laser to mid-IR pulses of 5 ns duration at 2940 nm wavelength. Beam steering and focusing was accomplished by gold coated mirrors (PF10-03-M01, Thorlabs, Newton, N.J.) and a 150 mm focal length CaF₂ lens (Infrared Optical Products, Farmingdale, N.Y.), respectively. At 5-6 mm downstream from the tip of the spray capillary, the laser beam having average output energy of 0.3 mJ/pulse was used to ablate the tissue sample at a right angle (90°). The laser spot size was determined by optical microscopy of the burn pattern produced on a photographic paper. The laser spot size had a 300 μm diameter.

According to certain embodiments, the electrospray system comprised a low-noise syringe pump (Physio 22, Harvard Apparatus, Holliston, Mass.) to feed a 50% methanol solution containing 0.1% (v/v) acetic acid through a stainless steel emitter with tapered tip having an outside diameter of 320 μm and an inside diameter of 50 μm. (MT320-50-5-5, New Objective Inc., Woburn, Mass.). Stable high voltage (2800 V) was generated by a regulated power supply (PS350, Stanford Research Systems, Inc., Sunnyvale, Calif.). The regulated power supply was directly applied to the emitter. The orifice of the sampling cone was on-axis with the electrospray emitter at a distance of 12 mm from its tip.

According to certain embodiments, the cells of interest were grown to populations to produce 10⁶ cells/pellet before subjecting the sample to LAESI-MS. The cells were washed twice with phosphate buffered saline (PBS) and pelleted by spindown (2000 rpm). The supernatant PBS was removed without disturbing the pellet, and 10 μL of the pellet was loaded onto a microscope slide and presented to the mass spectrometer for direct LAESI analysis. The microscope slide with the cell pellet was positioned 15 mm below the spray axis under ambient conditions (in air at ambient temperature and pressure). The microscope slide was mounted on a computer-controlled stepper motor-driven three-axis precision flexure stage (Nanomax TS, Thorlabs, Newton, N.Y.) for rastering and geometry optimization.

According to certain embodiments, the ion source was mounted on a Q-TOF Premier mass spectrometer (Waters, Milford, Mass.). Full scan mass spectra were recorded over the mass range of m/z 50-2,000 using a time-of-flight (TOF) analyzer at a resolution of 8,000 (FWHM). Individual measurements on the samples generally took a few seconds. For structure identification of individual metabolites, collision activated dissociation spectra was recorded by selecting the precursor ion using a quadrupole analyzer (transmission window 2 Da) and the product ions were resolved by the TOF analyzer. Argon was used as the collision gas at a collision cell pressure of 4×10⁻³ mbar and a collision energy set from 5 to 25 eV. Accurate masses were determined using an internal standard method. Glycine, methionine, N-acetyl phenylalanine, leucine enkephalin and glufibrinopeptide were dissolved in the electrospray solution to concentrations from 50 μM to 200 μM and used as internal standards. Averages of the LAESI spectra collected under similar experimental conditions for a fixed time were considered so that the approximate number of cells used for obtaining LAESI spectra were approximately the same for most of the samples.

The human metabolome database (HMDB; www.hmdb.ca), the MassBank high resolution mass spectral database (www.massbank.jp), the NIST/EPA/NIH mass spectral library, and the MetaCyc database (http://metacyc.org) were used with a mass tolerance ranging from 0.1 Da to 0.01 Da for the metabolite searches and identifications.

For verification purposes, according to certain embodiments, arginase activity was measured using the QuantiChrom Arginase Assay Kit (BioAssay Systems, Hayward, Calif.) according to the manufacturer's instructions. CEM and C81 cell lysates (10 μg and 100 μg) were measured in triplicates. The concentration of cAMP was measured using the CatchPoint Cyclic-AMP Fluorescent Assay Kit (Molecular Devices, Sunnyvale, Calif.) according to the manufacturer's instructions. CEM and C81 cell lysates (10 μg and 100 μg) were measured in triplicates. Glutathione reductase from CEM and C81 cell lysates (10 μg and 200 μg) were measured utilizing the Glutathione Reductase Assay Kit (Sigma, St. Louis, Mo.) according to the manufacturer's instructions.

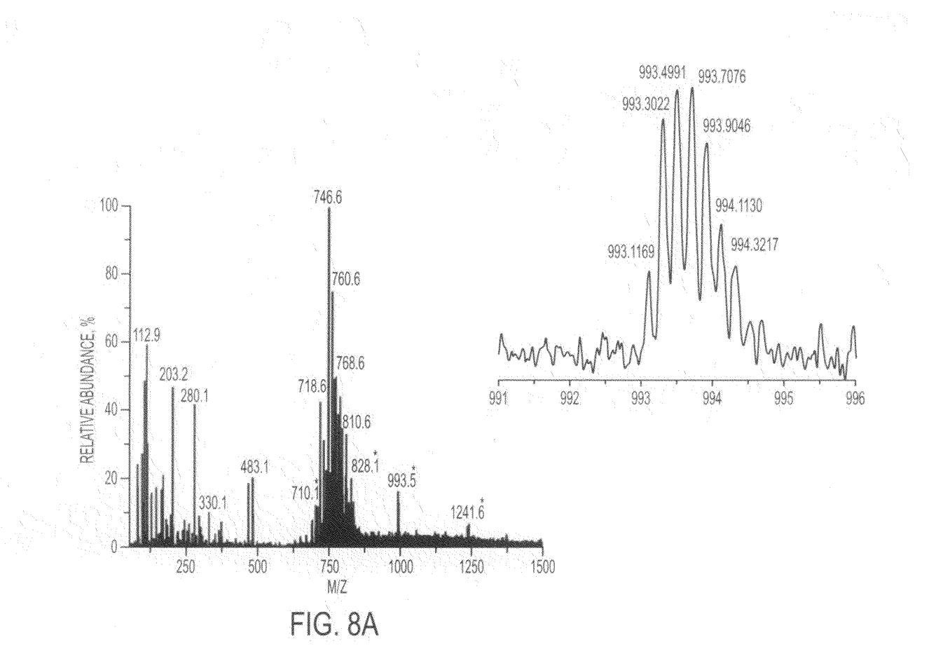

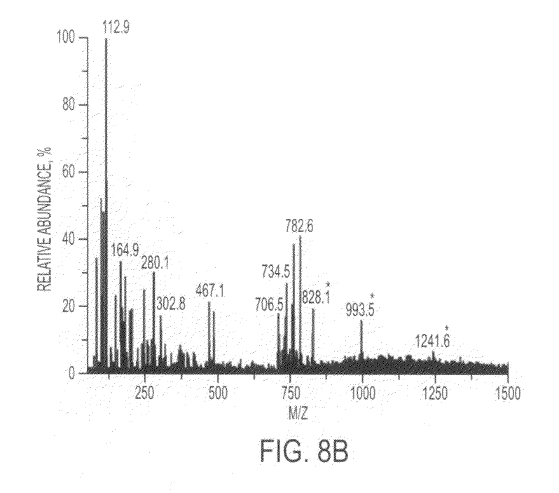

According to certain embodiments, the sample may comprise populations of uninfected (CEM) and HTLV1 infected (C81) T lymphocytes. These non-adherent cells were grown in an RPMI medium comprising inorganic salts, sugar, amino acids, vitamins and antibiotics. To minimize the interfering peaks from the medium in the LAESI spectra, the cells were washed with PBS and the cell pellet was loaded onto a microscope slide. The cells were directly ablated by multiple laser shots and the average LAESI spectra of 10-15 scans were used. The resulting positive ion spectra exhibited various cell related metabolite ions in the range of m/z 20-1500, but also included interfering peaks from PBS and the medium left in the cell pellet. Referring to FIG. 8, representative mass spectra according to certain embodiments are shown. The metabolite peaks observed in the mass spectra were identified based on the accurate masses, isotope distribution patterns, and structural information obtained from tandem MS. The background corrected spectra recorded from the cells consisted of protonated, sodiated, and potassiated species. The observed peaks may be due to small metabolites (<m/z 500), lipids (between m/z 690 and 850), and multiply charged peaks (between m/z 700 and 1300). Deconvolution of all multiply charged peaks (m/z 710, 828, 993 and 1241) showed correspondence to a single species with a nominal molecular weight of 4960.6, probably related to a peptide.

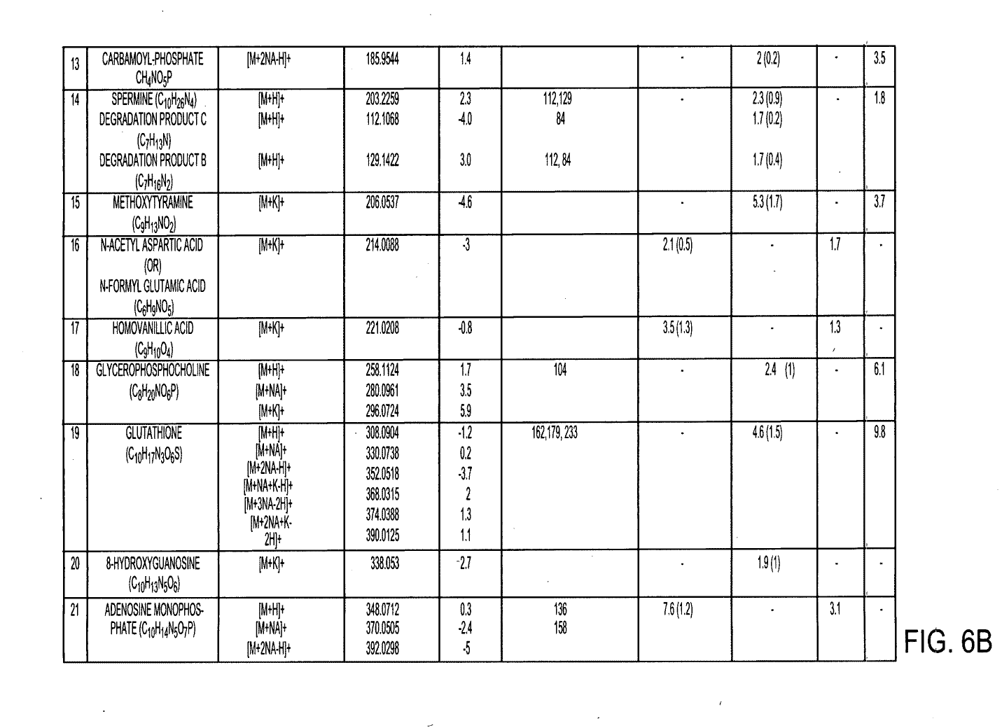

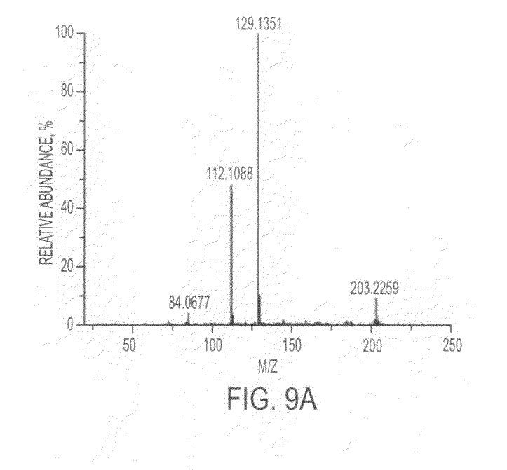

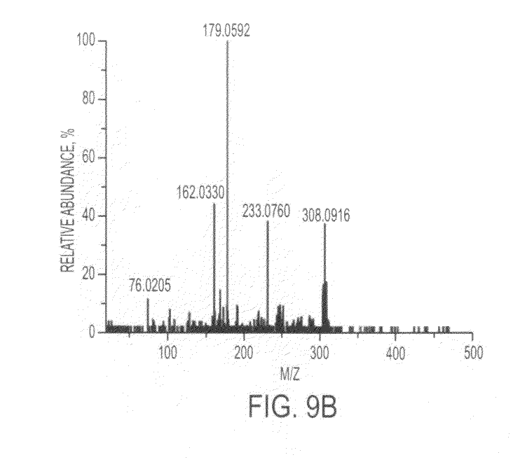

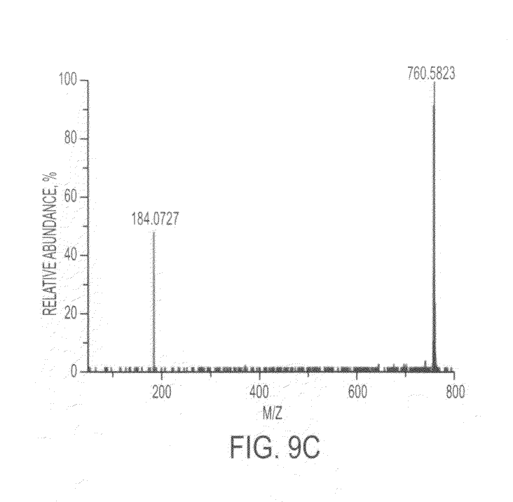

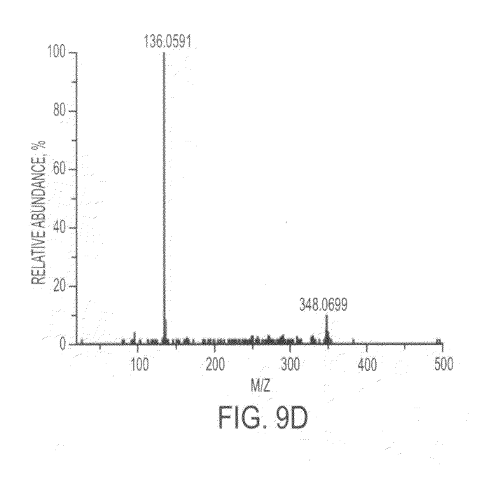

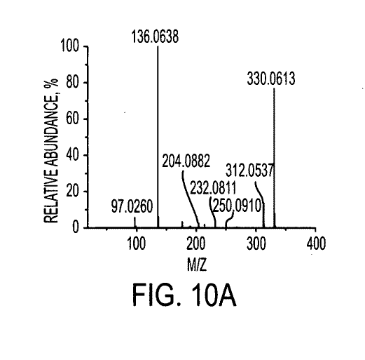

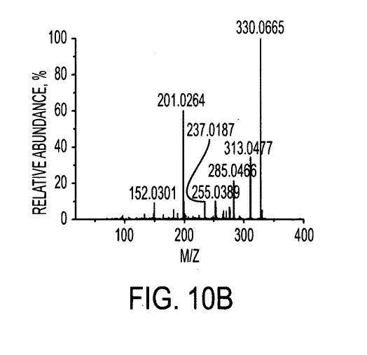

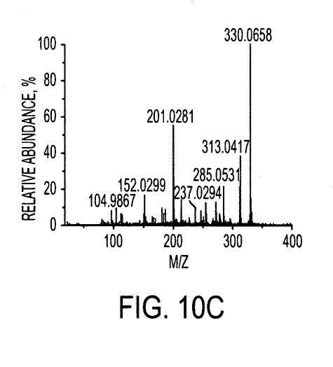

The spectra of CEM T lymphocytes and HTLV1 infected C81 T lymphocytes with Tax1 expression cells showed a similar set of ions, except for the lipid peaks, but consistent differences were identified in their relative ion yields. Referring to FIG. 6, a representative list of cell-specific metabolite ions and corresponding peak assignments, based on accurate mass and tandem mass spectral data and structure-specific fragment ions, is shown. The identification of the metabolites was confirmed by comparing their tandem mass spectra with the spectra of the corresponding standards from tandem MS databases. Spermine (m/z 203.2), glutathione (m/z 308.1), a phosphocholine lipid (PC(34:1), m/z 760.6) and adenosine monophosphate (m/z 348.1) were identified by the tandem mass spectra shown in FIGS. 9A-D. Both protonated cyclic AMP (cAMP) and sodiated glutathione may be theoretically assigned for the m/z 330.0738 ion. Distinguishing between these two ions may be difficult with the available mass resolution of m/Δm=10,000, even if both the ions are contributing to m/z 330 (as tested with standards). Referring to FIG. 10, sodiated glutathione may be identified by comparing the tandem mass spectrum of the m/z 330 ion from the T lymphocytes and the tandem mass spectrum of the m/z 330 ions generated from the two standards. However, cAMP at levels at or below the detection limit may contribute to the mass spectrum of the m/z 330 ion. In one experiment, cAMP in T lymphocytes was measured at levels of 6 pmoles/10⁷ cells. The interference from glutathione may cause difficulty in confirming the presence of cAMP by tandem MS.

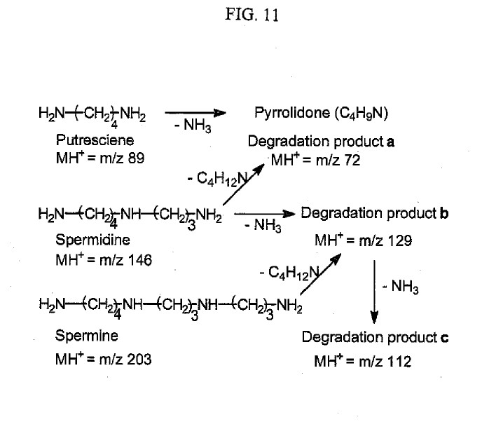

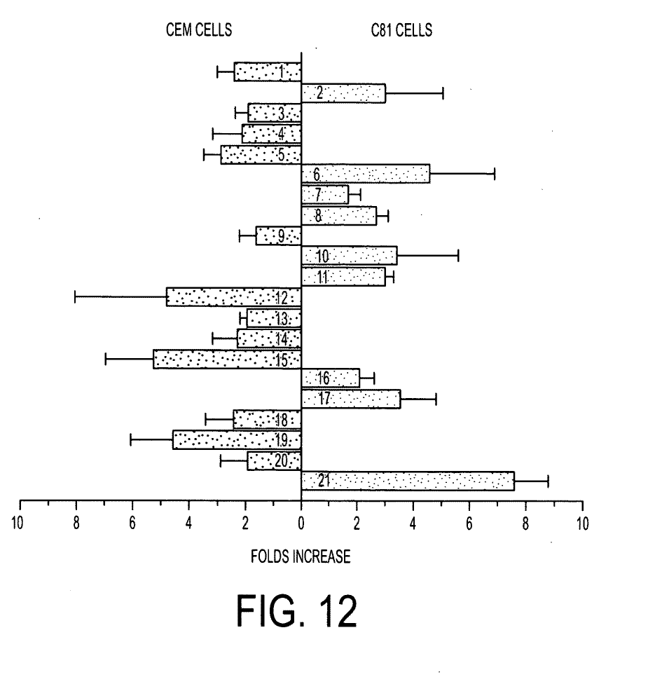

Representative mass spectra of degradation/fragmentation products of putrescine, spermidine and spermine are shown in FIG. 11 (designated as degradation products a, b and c). The degradation products a, b and c are shown in FIG. 6 at serial numbers 2 and 14. The formation of these products was confirmed by comparing the LAESI data from standard polyamines with those detected in T lymphocytes at similar experimental conditions. The m/z 72 ion primarily resulted from putrescine, and the m/z 129 and m/z 112 ions formed from both spermidine and spermine, probably mostly from the latter due to its higher ion yields. The abundances of these degradation products between the uninfected and infected cells track those of their precursors.

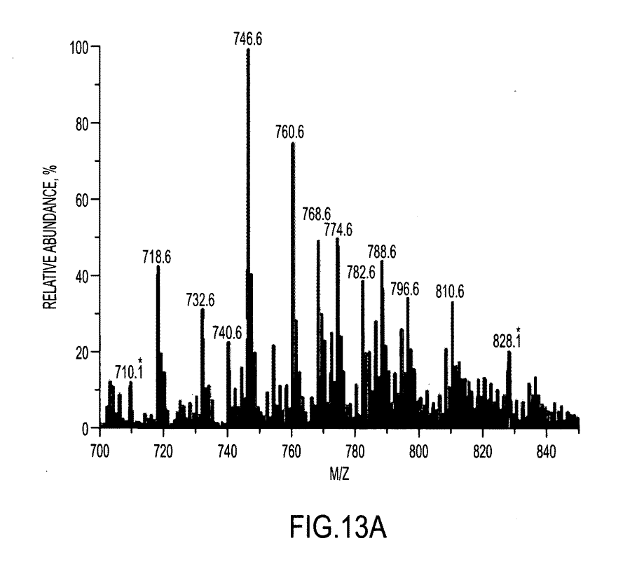

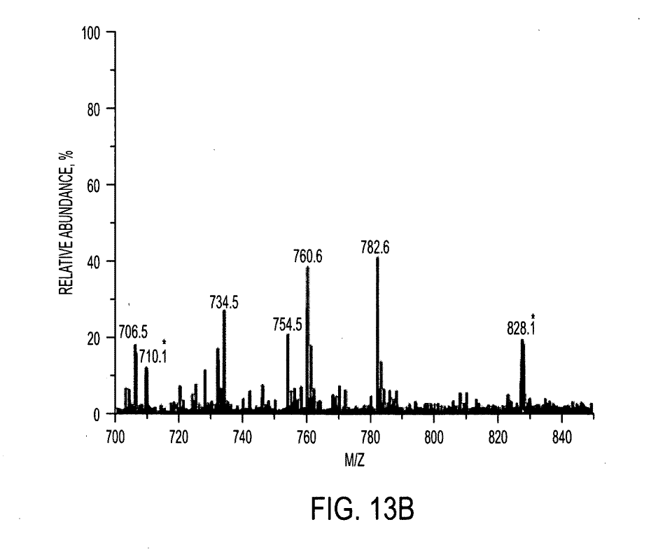

According to certain embodiments, the relative abundances of the detected ions in the mass spectra of non-HTLV1 transformed cells and HTLV1 transformed cells may be used to determine the extent of metabolic changes between them. The background peaks from PBS/medium solution were used as internal standards. The relative abundance ratios for each ion detected in CEM and C81 are listed in FIG. 6. Some metabolites may be detected as more than one ionic species (i.e., protonated, sodiated and potassiated). For example, glutathione was detected as six different ionic species. In such cases, the sum of the relative abundances of all the related species was used to calculate the abundance ratio. In case a particular peak is absent in a spectrum, the background (base line signal) was used to calculate the ratio. Upregulation may be measured by the abundance ratio of ions from HTLV1 transformed cells over non-HTLV1 transformed cells whereas downregulation may be measured by the inverse ratio. A ratio of 1 may signify a small change, if any. The changes in the levels of metabolites between CEM and C81 cells from triplicate experiments according to certain embodiments are shown in FIG. 13.

According to certain embodiments, the mass spectra of T lymphocytes comprised glucose (relative abundance <3%), the major component of the medium (11 mM). The mass of protonated spermine (203.2236) was close to that of sodiated glucose species (203.059), but these two peaks may be well separated. This, however, may raise the issue of possible contribution of medium-related peaks to the spectra detected from T lymphocytes. The mass spectrum of the medium alone showed that arginine (m/z 175), choline (m/z 104), and glutathione (m/z 308) contributed to the signal from the related metabolites in T lymphocytes. The glutathione and choline peaks were less than <2% with respect to the glucose peak (m/z 203, base peak), whereas the arginine peak was 25-30%. These ratios were consistent with values from diluted medium (100 times). The glucose peak appeared in both CEM and C81 cells (<3%) with an abundance ratio for m/z 203 close to unit value. The arginine peak that was negligible in CEM cells may be much higher than the glucose peak in C81 cells. This may confirm that the arginine interferences from the medium are negligible and the arginine levels are indeed upregulated in C81 cells.

According to certain embodiments, the relative mass spectra in the low mass region (<m/z 500) may be corrected for the medium and electrospray related background. After correction, 43 ions were exclusively related to T lymphocytes, and 37 of these ions corresponded to the 21 metabolites shown in FIG. 6. The unassigned ions showing variations in their relative abundances between the non-HTLV1 transformed cells and HTLV1 transformed cells were at m/z 158.1572 (abundance ratio C81/CEM=2.5), 228.0363 (abundance ratio C81/CEM=2.6), 260.0298 (abundance ratio C81/CEM=3.6), 311.9216 (abundance ratio C81/CEM=1.5), 333.9604 (abundance ratio C81/CEM=2.2), and 346.0616 (abundance ratio C81/CEM=1.2). As shown in FIG. 6, many metabolites were downregulated in the HTLV1 transformed cells, e.g., spermine, choline, phosphocholine, glycerophosphocholine, and glutathione, and many other metabolites were upregulated in the infected cells, e.g., pyrrolidine, creatine, arginine, dopamine and adenosine monophosphate.

According to certain embodiments, representative mass spectra of the T lymphocytes may comprise glycerophosphocholine (PC) lipids. Referring to FIG. 13, significant changes were observed in the lipid abundances and types between non-HTLV1 transformed cells and HTLV1 transformed cells. Tandem mass spectra of all major lipid peaks yielded a single product ion at m/z 184 (a typical spectrum of the m/z 760.6 ion is shown in FIG. 9C) to confirm PC lipids.

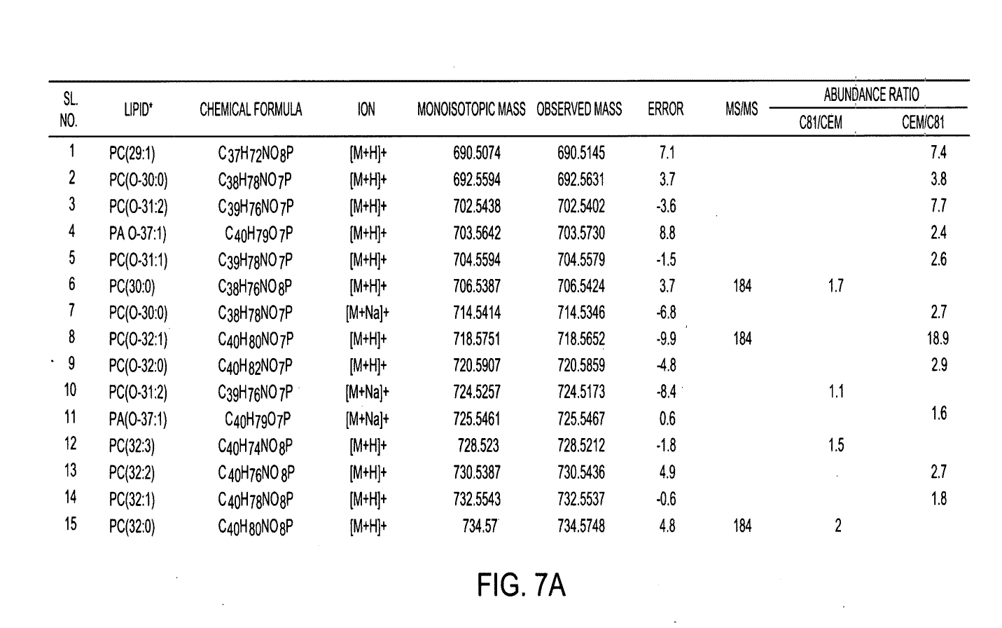

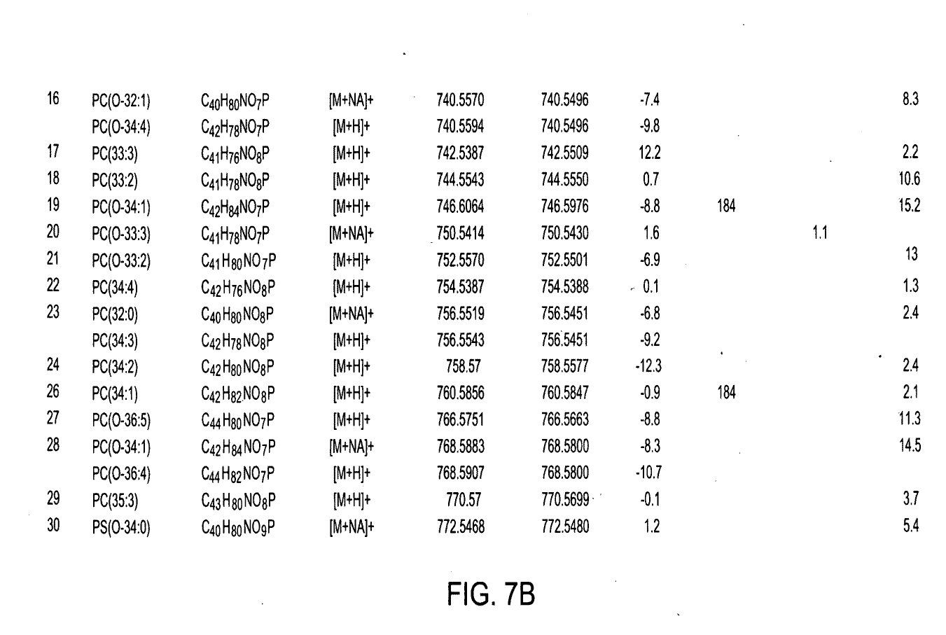

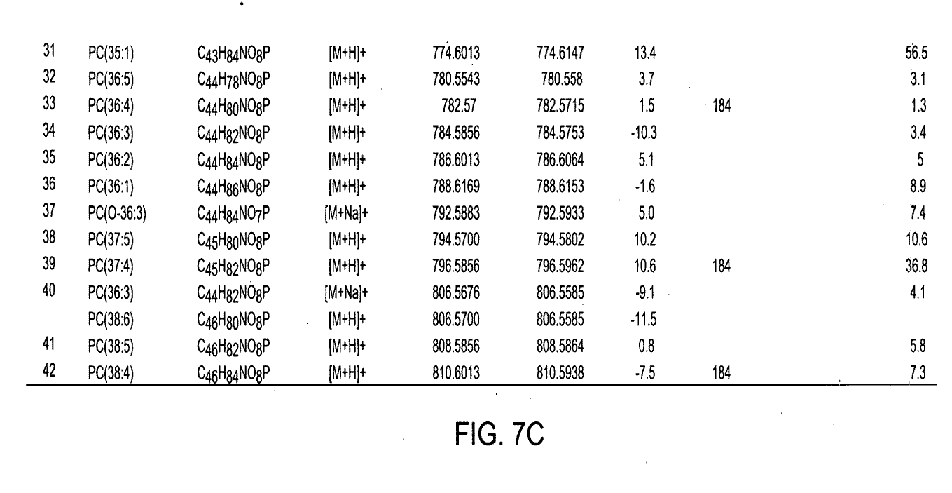

As shown in FIG. 7, the lipid peaks were assigned based on tandem mass spectrometric and accurate mass information. FIG. 7 includes diacyl glycerophosphocholines (PC(Cₙ:dbn), where Cₙ represents the total number of carbons and dbn represents the total number of double bonds in the two fatty acid side chains and the alkylacyl/alkenylacyl glycerophosphocholines or plasmalogens, (PC(O—Cₙ:dbn)). FIG. 7 also includes the relative abundance ratio values. Most of the lipids detected in non-HTLV1 transformed cells were downregulated in HTLV1 transformed cells. Only a few lipids were retained in the HTLV1 transformed cells, and the levels of PC(30:0), PC(0-31:2), PC(32:3), PC(32:0), and PC(0-33:3) were higher compared to the non-HTLV1 transformed cells. Collision induced dissociation (CID) products from in-source fragmentation/degradation of lipids appeared at ions m/z 184 and 104. When the mass spectrum is recorded for a standard lipid (PC(16:0/18:1)) under similar experimental conditions, the fragment ions m/z 104 and 184 were marginally observed (<0.5%). This confirmed that the detected choline peaks corresponded to metabolites and not to CID artifacts.

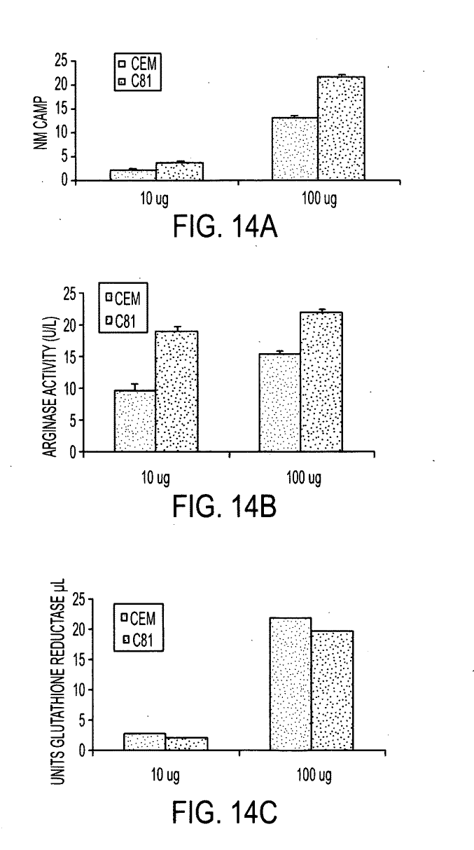

According to certain embodiments, the changes in metabolite levels between the non-HTLV1 transformed T lymphocytes and HTLV1 transformed T lymphocytes may be verified at the protein level by quantifying the enzymes or proteins involved in the related metabolic pathway. The levels of cAMP, arginase, and glutathione reductase in CEM and C81 cells were measured using biochemical assays. Referring to FIG. 14, arginase and cAMP levels were upregulated and glutathione reductase levels were downregulated in the HTLV1 transformed cells (C81) as compared to non-HTLV1 transformed cells (CEM). The assays correlate with the changes detected by LAESI-MS in the corresponding metabolites.

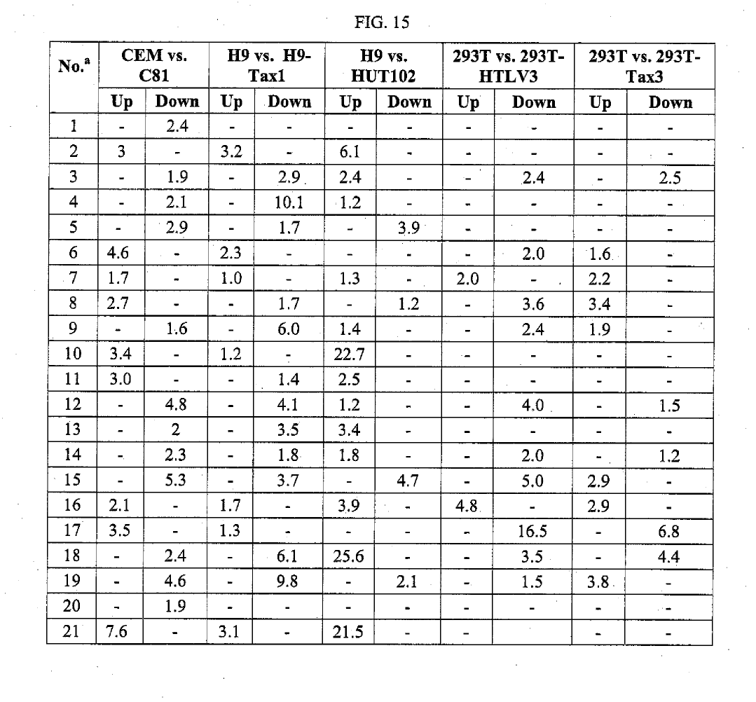

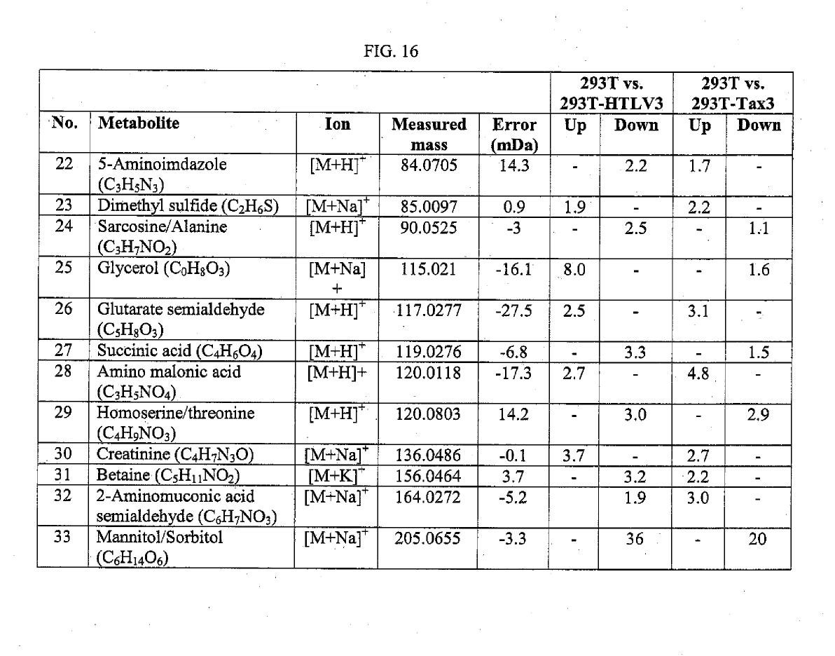

According to certain embodiments, the sample may comprise other cell lines, e.g., non-HTLV1 transformed T lymphocytes (H9), their Tax1-transfected counterparts (H9-Tax1), and HTLV1 transformed cells (HUT102 cells). The metabolic changes upon transfection are listed in FIG. 16. Referring to FIG. 15, metabolite abundance ratios indicating up or down regulation for HTLV1 infected T cells (C81, HUT102) and HTLV3, Tax1 or Tax3 transformed cells (293-HTLV3, H9-Tax1, 293-Tax3, respectively). The metabolites in these cells were compared to the CEM and C81 cells. The pattern of upregulation and downregulation of metabolites, such as glutathione and adenosine monophosphate, was similar to the CEM/C81 case. These results suggest that the metabolic changes observed in the HTLV1 infected cells may be partly attributed to Tax1 expression.

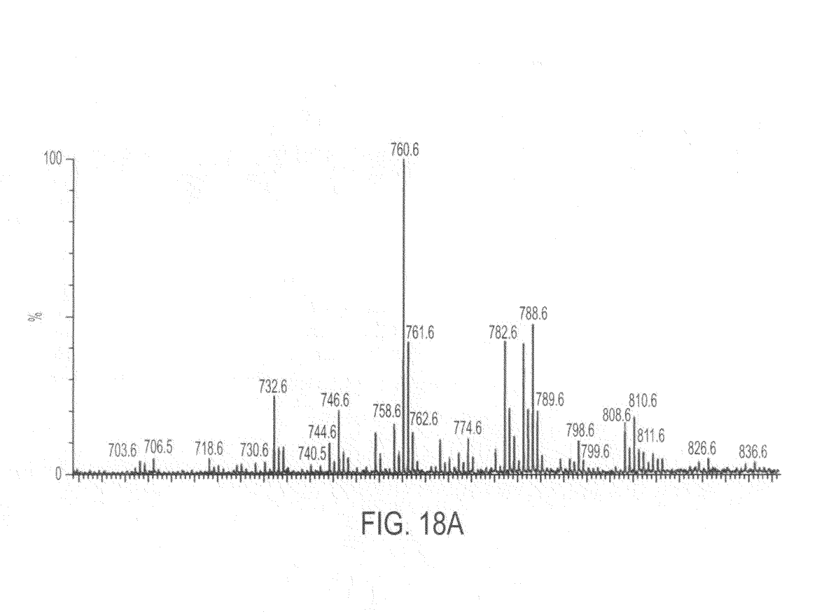

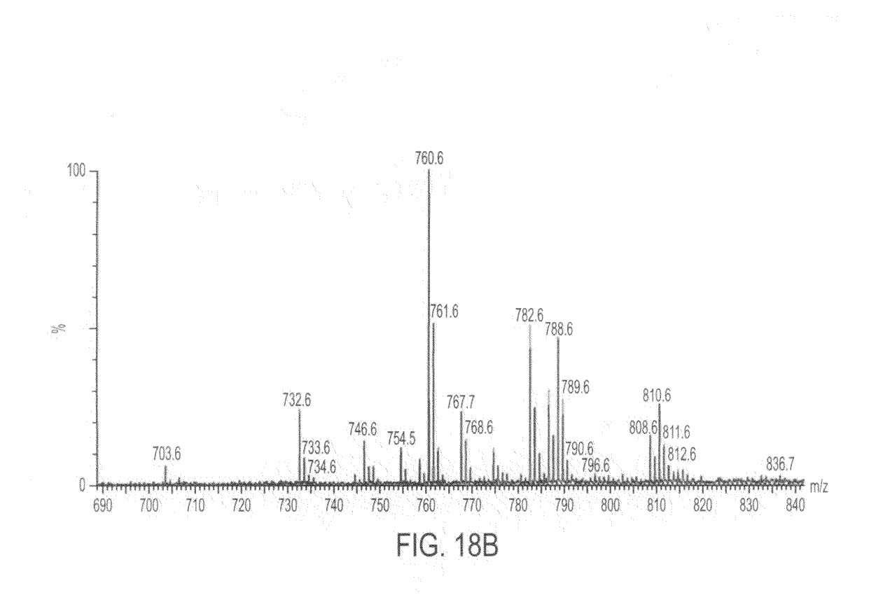

According to certain embodiments, the sample may comprise other cell lines to determine the specificity of the observed metabolite changes to HTLV1 transformation, e.g., uninfected 293T kidney epithelial cells, and on HTLV3 and Tax3 transfected 293T cells. The metabolic changes upon HTLV1 transformation and the presence of Tax3 are listed in FIG. 15. Referring to FIG. 15, metabolites detected in 293T, 293T-HTLV3 and 293T-Tax3 cells, and their abundance ratios indicating up and down regulation due to HTLV3 transfection or the presence of Tax3. As shown in FIGS. 15 and 16, the 293T cells showed a variety of ions that were not detected in the CEM, C81, H9 and HUT102 cells, but there were some metabolites common to all these cells. The observed changes for HTLV3 and Tax3 transfected 293T cells did not match those found in HTLV1 transformed cells. As shown in FIG. 18, the lipid peaks that showed prominent changes in the HTLV1 transformed cells were found to be unaltered in HTLV3/Tax3 affected 293T cells.

According to certain embodiments, multiple abundant ionic species of glutathione may be detected in non-HTLV1 transformed T lymphocytes reflecting high concentrations, whereas in HTLV1 transformed cells there may be a 2 to 5-fold decrease in their abundance. The reduced form of glutathione (GSH) may be the most predominant thiol present in mammalian cells with concentrations up to 12 mM. GSH may serve several important functions, such as antioxidant (protection against oxidative stress), cofactor in isomerization reactions, transport and storage form of cysteine, and regulator of intracellular redox status, cell proliferation and apoptosis. Biologically, the oxidized glutathione (GSSG) may be converted to GSH by the enzyme glutathione reductase. The ratio of GSH and GSSG may serve as a representative indicator of the antioxidative capacity of the cell. Cellular GSH concentrations may be reduced in response to protein malnutrition, oxidative stress, and other pathological conditions. Intracellular GSH levels may regulate T lymphocyte function, and deficiency of GSH may be associated with HIV infection.

According to certain embodiments, the GSH level in HTLV1 transformed T lymphocytes was decreased. The levels of glutathione reductase in both non-HTLV1 transformed cells and HTLV1 transformed cells were measured using an enzyme assay, and GSH was reduced in HTLV1 transformed cells and GSH was reduced in HTLV3 transformed 293T cells.

According to certain embodiments, the sample may comprise spermine, spermidine and putrescine, which belong to the polycationic compounds named polyamines. These polyamines may be involved in genetic processes, such as DNA synthesis and gene expression, and play a major role in cell proliferation, cell differentiation, and programmed cell death. Referring to FIG. 16, the biosynthesis of polyamines is tightly regulated in cells, and ornithine in the urea cycle is their precursor. The level of these polyamines may indicate the actual condition of the cell, including whether the cell is virally infected. In one experiment, spermine levels were higher in all of the transformed cells except for the case of HUT102 and the putrescine level and spermidine level, the precursors of spermine, were upregulated in HTLV1 transformed cells. Spermidine was also upregulated in the 293T-HTLV3 and the 293T-Tax3 cell lines. Without wishing to be bound to any particular theory, this may indicate that the viruses and the Tax transformation affect the tightly regulated biosynthesis of polyamines in the cells, thereby causing disturbances in the genetic processes. Although the trends for individual amines were not completely consistent among HTLV1 transformed cells, the effect of viruses on the overall polyamine biosynthesis is reflected. In one experiment, arginine that was converted into ornithine, the precursor of polyamines in the urea cycle, was upregulated in HTLV1 transformed cells, and particularly high in HUT102. Spermine, spermidine and putrescine may associate with nucleic acids due to electrostatic interactions between the positively charged ammonium groups of the polyamines and the negatively charged phosphates of nucleic acids.

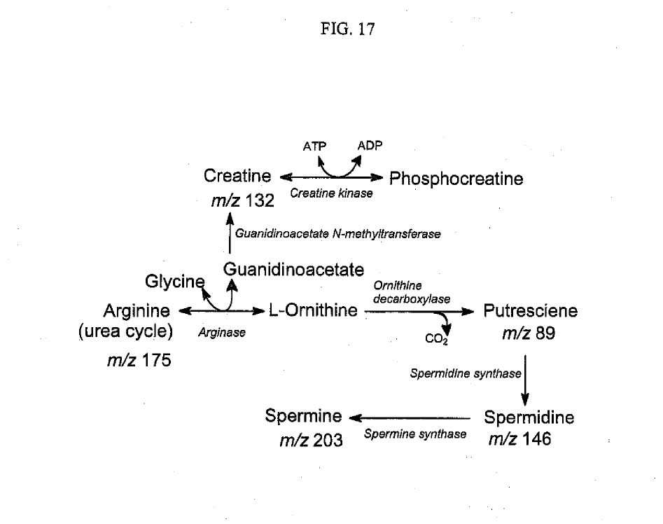

According to certain embodiments, the arginine levels in HTLV1 transformed cells were increased. The transcriptional upregulation of arginase in infected cells, which may be confirmed by the enzyme assay, was consistent with elevated levels of arginine upon viral infection. The role of arginine may play a role in the survival of endothelial cells during oxidative stress. Deprivation of arginine may cause serious disturbances in cellular function and enhances apoptosis. Arginine availability may contribute to the regulation of T lymphocyte function in cancer. Referring to FIG. 17, arginine is a precursor in the biosynthesis of creatine, and may be an important molecule in energy supply. The blood of HTLV2 infected patients may have abnormal creatine phosphokinase levels. The enzymes related to creatine and arginine metabolism were found to be significantly upregulated in malignant cells. Accordingly, a finding of upregulation of arginine levels in HTLV1 transformed cells is in line with other biological systems.

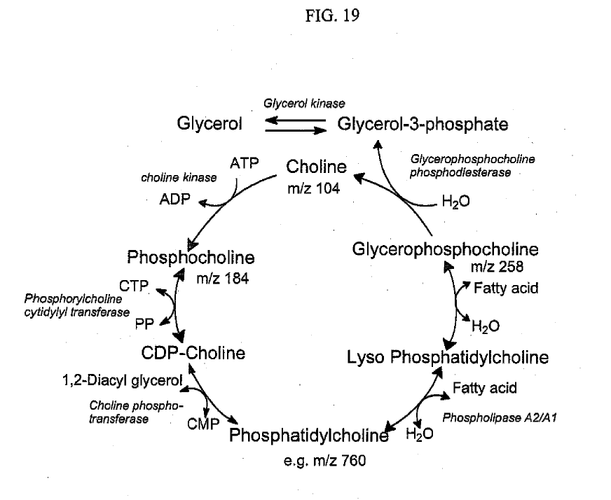

According to certain embodiments, the mass spectra of T lymphocyte cells and kidney epithelial cells show that choline containing metabolites, e.g., choline, phosphocholine, glycerophosphocholine, and several glycerophosphocholine lipids, may be downregulated upon transformation by HTLV1, HTLV3, Tax1 or Tax3 when compared to non-transformed cells, except in the HUT102 case. Referring to FIG. 19, choline containing metabolites may have a role in lipid metabolism. Choline may be a precursor of various metabolites and the intracellular routing of choline to its various metabolic pathways, phosphorylation, oxidation, and acetylation may be cell specific. Choline and choline metabolites may be regenerated by controlled breakdown of choline phospholipids through several pathways. Increases in choline containing metabolites may be associated with a number of disorders, including malignant cell growth. Changes in the lipid levels in HIV and HCMV infected cells may be detected using proteomic and metabolomic platforms, respectively. Fatty acid biosynthesis in infected cells may also be considered as an antiviral response. An increase in the levels of choline containing metabolites may be associated with a number of disorders, for example, substantial upregulation of fatty acid synthesis in HCMV infected fibroblast cells.

In vivo NMR spectroscopy may be used to monitor choline containing metabolites, however, it may be difficult to determine which specific metabolites are altered. According to certain embodiments, choline containing metabolites may be directly subjected to LAESI-MS to determine which specific metabolites are altered. The mass spectra of non-transformed cells and transformed cells may provide information about the precursors and lipid components simultaneously. In one experiment, a decrease in the glycerophosphocholine lipid content in HTLV1 transformed cells confirmed increased lipid catabolism to produce fatty acids. Apart from a few lipids (PC(30:0), PC(32:5), PC(32:3), PC(32:0), and PC(34:6) that remained at higher levels in HTLV1 transformed cells, most of the glycerophosphocholine lipids present in non-HTLV1 transformed cells were downregualated in the HTLV1 transformed cells.

Levels of phosphorylated adenosine nucleotides, including ATP, ADP, and AMP, may define the energy state in living cells. Quantitation of individual adenine nucleotides may be used for the assessment of the energy state of cells. The level of exogenous ATP in the body may be increased in various inflammatory and shock conditions. Extracellular ATP may be important for cell-to-cell communication and in the immune system. In one experiment, AMP was detected directly from cells subjected to LAESI-MS. Significantly elevated AMP abundance was observed in HTLV1 and Tax1 transformed cells. Referring to FIG. 19A, AMP may be formed by the dephosphorylation of ATP/ADP or by the hydrolysis of cAMP. Apart from being a degradation product of ATP, AMP may activate the AMP-activated kinase (AMPK) system that is ubiquitously expressed in mammalian cells. Without wishing to be bound to any particular theory, it may be involved in the response to a variety of metabolic stresses that disturb the cellular energy homeostasis.

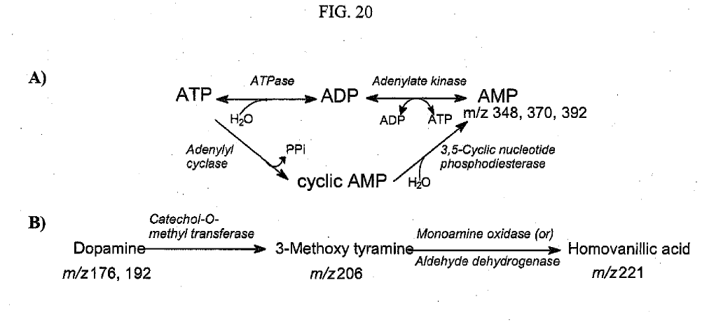

According to certain embodiments, the sample may comprise cAMP. cAMP is a second messenger and activates several protein kinases that may be involved in significant biochemical processes. The amount of cAMP known to be present in T lymphocytes is 6 pmol/10⁷ cells. In one experiment, glutathione interfered with the detection of cAMP. Referring to FIG. 14, cAMP was measured by an immunoassay. Referring to FIG. 20A, the cAMP levels (adenylyl cyclase activity) were increased in HTLV1 transformed cells compared with non-HTLV1 transformed cells. The changes in cAMP levels may indicate HIV and in HTLV1 infected T lymphocytes.

According to certain embodiments, the sample may comprise dopamine, a neuromodulator, and its metabolites, methoxytyramine and homovanillic acid, in T lymphocytes. Dopamine belongs to the group of catecholamines, and may be involved in the neuroimmunological network. T lymphocytes may be activated by neurotransmitters via neurotransmitter receptors that may elicit crucial functions. Catecholamines may be synthesized in mouse lymphocytes. Increased levels of catecholamines may indicate an activated state. As shown in FIG. 20B, dopamine may be biosynthesized in the body from tyrosine, and related metabolic pathways. In one experiment, dopamine and homovanillic acid levels were upregulated and methoxytyramine was downregulated in HTLV1 transformed cells.

All documents cited herein are, in relevant part, incorporated herein by reference, but only to the extent that the incorporated material does not conflict with existing definitions, statements, or other documents set forth herein. To the extent that any meaning or definition of a term in this document conflicts with any meaning or definition of the same term in a document incorporated by reference, the meaning or definition assigned to that term in this document shall govern. The citation of any document is not to be construed as an admission that it is prior art with respect to this document.

While particular embodiments of mass spectrometers and methods of making and using the same have been illustrated and described, it would be obvious to those skilled in the art that various other changes and modifications can be made without departing from the spirit and scope of the invention. Those skilled in the art will recognize, or be able to ascertain using no more than routine experimentation, numerous equivalents to the specific apparatuses and methods described herein, including alternatives, variants, additions, deletions, modifications and substitutions. This disclosure including the appended claims is therefore intended to cover all such changes and modifications that are within the scope of this invention.

1. A method of mass spectrometry comprising:
subjecting a sample comprising an indicator to laser ablation electrospray ionization mass spectrometry;
determining a relative intensity of the indicator; and
comparing the relative intensity of the indicator to a standard indicator intensity.

2. The method of claim 1 comprising classifying the sample as belonging to or not belonging to the standard indicator intensity.

3. The method of claim 2, wherein the indicator comprises at least one biomarker.

4. The method of claim 3, wherein the at least one biomarker is related to a disease state.

5. The method of claim 4, wherein not belonging to the standard indicator intensity indicates that the sample is predicted to comprise the disease state.

6. The method of claim 4, wherein the indicator comprises a plurality of indicators, and determining the relative intensity of each indicator to form a sample metabolite pattern, comparing the sample metabolite pattern to a standard metabolite pattern comprising the standard indicator intensity of each of the plurality of indicators, and classifying the sample as belonging to or not belonging to the standard metabolite pattern.

7. The method of claim 6, wherein not belonging to the standard metabolite pattern indicates that the sample is predicted to comprise the disease state.

8. The method of claim 5, wherein the disease state is associated with at least one of a viral infection, a bacterial infection, and a metabolic disorder.

9. The method of claim 5, wherein the indicator is selected from the group consisting of metabolites, lipids, lipid precursors, lipid components, nucleic acids, proteins, peptides, carbohydrates, and combinations thereof.

10. The method of claim 5, wherein the disease state is at least one of human immunodeficiency virus, human T-lymphotropic virus type 1, human T-lymphotropic virus type 3, and the indicators are selected from the group consisting of glutathione, spermine, spermidine, putrescine, arginine, creatine, choline, phosphocholine, glycerophosphocholine, glycerophosphocholine lipids, ATP, ADP, AMP, cAMP, dopamine, dopamine metabolites, and any combination thereof.

11. The method of claim 1, wherein the sample is selected from the group consisting of a single cell, cells, biofilms, and tissues.

12. The method of claim 11, wherein the single cell has a smallest dimension from 5 micrometers to 50 micrometers.

13. The method of claim 1, wherein subjecting to laser ablation electrospray ionization mass spectrometry comprises:
ablating the sample with an infrared laser under ambient conditions to form an ablation plume;
intercepting the ablation plume by an electrospray plume; and
detecting the indicator by mass spectrometry.

14. The method of claim 1, wherein subjecting to laser ablation electrospray ionization mass spectrometry excludes pretreating the sample with a matrix material.

15. The method of claim 1, wherein the standard indicator intensity comprises at least one of an internal reference and an external reference.

16. An in situ method of determining a metabolic state of a sample comprising an indicator, the method comprising:
ablating the sample with an infrared laser under ambient conditions to form an ablation plume;
intercepting the ablation plume by an electrospray plume;
detecting the indicator by mass spectrometry;
determining a relative intensity of the indicator;
comparing the relative intensity of the indicator to a standard indicator intensity; and
classifying the sample as belonging to or not belonging to the standard indicator intensity.

17. The method of claim 16 comprising:
ablating a second sample comprising the indicator with an infrared laser under ambient conditions to form a second ablation plume;
intercepting the second ablation plume by a second electrospray plume;
detecting the indicator of the second sample by mass spectrometry;
determining a relative intensity of the indicator of the second sample;
comparing the first indicator and second indicator to at least one of each other and the standard indicator intensity; and
classifying at least one of the first sample and the second sample as belonging to or not belonging to the standard indicator intensity.

18. The method of claim 17, wherein the indicator comprises intracellular metabolites from a single cell.

19. The method of claim 17, wherein not belonging to the standard indicator intensity indicates metabolic changes in the sample associated with a disease state.

20. The method of claim 16, wherein the metabolic state is selected from the group consisting of stages of a cell cycle, developmental stages, environments, nutritional supplies, taxonomic units, genetic units, infected and uninfected states, diseased and healthy states, and different stages of a pathogenicity.
